(19) United States
(12) Patent Application Publication
(10) Pub. No.: US 2025/0143900 A1
(43) Pub. Date: May 8, 2025

(54) PROSTHETIC VALVE PREPARATION ASSEMBLIES AND ASSOCIATED METHODS

(71) Applicant: EDWARDS LIFESCIENCES CORPORATION, Irvine, CA (US)

(72) Inventors: Grant Jason Gangeness, Carlsbad, CA (US); Hannah Reed Bettencourt, Huntington Beach, CA (US); James William Ventress, Lake Havasu City, AZ (US)

(21) Appl. No.: 19/014,738

(22) Filed: Jan. 9, 2025

Related U.S. Application Data
(63) Continuation of application No. PCT/US2023/027476, filed on Jul. 12, 2023.
(60) Provisional application No. 63/368,632, filed on Jul. 15, 2022.

(51) Int. Cl. A61F 2/95; A61F 2/24

(52) U.S. Cl. CPC A61F 2/9524; A61F 2/2427

(57) ABSTRACT

Prosthetic valve preparation assemblies and associated methods are disclosed herein. A crimper assembly can include a first crimp stop and a second crimp stop that define respective ranges of motion of an actuator handle. A method of mounting an implantable prosthetic valve to a valve delivery apparatus can include inserting a balloon catheter within the implantable prosthetic valve, partially crimping an annular frame of the implantable prosthetic valve, positioning a crimp alignment tool in an alignment configuration relative to the valve delivery apparatus, positioning the implantable prosthetic valve to engage the crimp alignment tool, and fully crimping the annular frame.

### CROSS REFERENCE TO RELATED APPLICATION

This application is a continuation of PCT Patent Application No. PCT/US2023/027476 filed Jul. 12, 2023, which claims the benefit of U.S. Provisional Application No. 63/368,632, filed Jul. 15, 2022, each of these applications being incorporated by reference herein in its entirety.

### FIELD

The present disclosure relates to prosthetic valve preparation assemblies and associated methods, and more particularly to assemblies and methods that utilize a device for crimping an implantable prosthetic valve onto a delivery apparatus.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Disclosed herein are prosthetic valve preparation assemblies including crimper assemblies, valve delivery assemblies, implantable prosthetic valves, and crimp alignment tools, as well as associated methods. The disclosed prosthetic valve preparation assemblies and methods can, for example, ensure that the implantable prosthetic valve is crimped to the valve delivery apparatus at a desired location.

A crimper assembly can comprise an actuator handle, a second crimp stop, and a first crimp stop. The actuator handle is configured to be manually actuated to crimp an implantable prosthetic valve around a valve delivery apparatus. The valve delivery apparatus can comprise a handle and one or more shafts coupled to the handle. The second crimp stop defines a full range of motion of the actuator handle, and the first crimp stop defines a partial range of motion of the actuator handle. The first crimp stop is configured to be selectively and operatively coupled to the second crimp stop.

In a representative example, the crimper assembly further comprises a base, a housing fixedly mounted to the base and defining a crimper assembly central axis, and a plurality of circumferentially arrayed nesting jaws supported within the housing. The jaws are radially moveable within the housing toward and away from the central axis. The plurality of jaws collectively define a crimper aperture that varies in diameter as the jaws move toward and away from the central axis.

In some examples, the actuator handle is moveable relative to the housing to move the jaws toward and away from the central axis.

In some examples, when the first crimp stop is operatively coupled to the second crimp stop, actuating the actuator handle to engage the first crimp stop operates to bring the crimper aperture to a first stop diameter.

In some examples, when the first crimp stop is removed from the second crimp stop, actuating the actuator handle to engage the second crimp stop operates to bring the crimper aperture to a second stop diameter that is less than the first stop diameter.

In some examples, a crimper assembly comprises one or more of the components recited in Examples 1-14 and/or 25-43 below.

A method of mounting an implantable prosthetic valve to a valve delivery apparatus can comprise inserting a balloon catheter of the valve delivery apparatus within a frame central passage of the implantable prosthetic valve, partially crimping an annular frame of the implantable prosthetic valve with a crimper assembly, and fully crimping the annular frame of the implantable prosthetic valve.

In some examples, a method further comprises operatively coupling a crimp alignment tool to a distal portion of the valve delivery apparatus to position the crimp alignment tool in an alignment configuration relative to the valve delivery apparatus and positioning the implantable prosthetic valve to engage the crimp alignment tool.

In some examples, the crimper assembly includes an actuator handle configured to be manually actuated to crimp the implantable prosthetic valve around the valve delivery apparatus, a second crimp stop that defines a full range of motion of the actuator handle, and a first crimp stop that defines a partial range of motion of the actuator handle and that is configured to be selectively and operatively coupled to the second crimp stop.

In some examples, the partially crimping the annular frame comprises, with the first crimp stop operatively coupled to the second crimp stop, actuating the actuator handle to engage the first crimp stop.

In some examples, the fully crimping the annular frame comprises actuating the actuator handle to engage the second crimp stop.

In some examples, the method further comprises, subsequent to the partially crimping the annular frame and prior to the fully crimping the annular frame, removing the first crimp stop from the second crimp stop.

In some examples, a method comprises one or more of the steps recited in Examples 19-24 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view illustrating components of a prosthetic valve preparation assemblies according to one example.

FIG. 2 is a detail perspective view of an example of a crimp alignment tool and a balloon cover assembly installed on a valve delivery apparatus.

FIG. 3 is an exploded view of the crimp alignment tool, the balloon cover assembly, and the valve delivery apparatus of FIG. 2

FIG. 4 is a partial perspective view illustrating the crimp alignment tool installed on the valve delivery apparatus and the balloon cover assembly removed from the valve delivery apparatus.

FIG. 5A is an isometric view of a pair of balloon cover clamshell portions in an assembled configuration.

FIG. 5B is a side view of one of the balloon cover clamshell portions of FIG. 5A.

FIG. 5C an isometric view of one of the balloon cover clamshell portions of FIG. 5A.

FIG. 5D is an isometric view of another balloon cover clamshell portion of FIG. 5A.

FIG. 6A is an isometric view of the crimp alignment tool.

FIG. 6B is another isometric view of the crimp alignment tool of FIG. 6A.

FIG. 6C is a plan view of the crimp alignment tool of FIG. 6A.

FIG. 6D is a cross-sectional view of the crimp alignment tool of FIG. 6A, as viewed along the line 6D-6D in FIG. 6C.

FIG. 7 is a side view of a crimp stop assembly according to one example.

FIG. 8A is a side view of a first crimp stop of the crimp stop assembly of FIG. 7.

FIG. 8B an isometric view of the first crimp stop of FIG. 8A.

FIG. 8C is another isometric view of the first crimp stop of FIG. 8A.

FIG. 9A is a side view of a second crimp stop of the crimp stop assembly of FIG. 7.

FIG. 9B is an isometric view of the second crimp stop of FIG. 9A.

FIG. 9C is another isometric view of the second crimp stop of FIG. 9A.

FIG. 10 is a side view of another example of a crimp stop assembly.

FIG. 11 is a perspective view of an example of an implantable prosthetic valve.

FIG. 12 is a perspective view of an example of an implantable prosthetic valve in an operational configuration and positioned on an example of a valve delivery apparatus.

FIG. 13 is a perspective view of the implantable prosthetic valve and the valve delivery apparatus of FIG. 12 positioned in a crimper assembly.

FIG. 14 is a perspective view of the implantable prosthetic valve, the valve delivery apparatus, and the crimper assembly of FIG. 13 with an actuator handle of the crimper assembly engaging a first crimp stop of the crimper assembly.

FIG. 15 is a perspective view of the implantable prosthetic valve and the valve delivery apparatus of FIG. 12 with the implantable prosthetic valve in an intermediate configuration and engaging an example of a crimp alignment tool.

FIG. 16 is a perspective view of the implantable prosthetic valve, the valve delivery apparatus, and the crimper assembly of FIG. 15 with the actuator handle of the crimper assembly of FIG. 13 engaging a second crimp stop of the crimper assembly.

FIG. 17 is a perspective view of the implantable prosthetic valve and the valve delivery apparatus of FIG. 12 with the implantable prosthetic valve in a delivery configuration.

FIG. 18 is a perspective view of the implantable prosthetic valve and the valve delivery apparatus of FIG. 17 with a stylet of the valve delivery apparatus removed.

FIG. 19 is a flowchart depicting examples of methods of mounting an implantable prosthetic valve to a valve delivery apparatus.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined. Further, the term “radial” refers to a direction that is arranged perpendicular to the axis and points along a radius from a center of an object (where the axis is positioned at the center, such as the central longitudinal axis of the delivery assembly).

As used herein, “e.g.” means “for example,” and “i.e.” means “that is.”

### Introduction to the Disclosed Technology

Disclosed herein are various systems, apparatuses, methods, etc., including prosthetic valve preparation assemblies that can be used in conjunction with implantable prosthetic valves (e.g., transcatheter heart valves (THV)). The various systems, apparatuses, methods, etc. disclosed herein generally are directed to preparing the implantable prosthetic valves for delivery through a patient's vasculature to a target implantation site. In particular, the present disclosure pertains to examples in which the implantable prosthetic valve comprises an annular frame that can be crimped to a constricted diameter for passage through the patient's vasculature and subsequently expanded to an operative diameter by an inflatable balloon of the valve delivery apparatus.

Before crimping, a balloon-expandable implantable prosthetic valve is typically placed over an expandable balloon on a catheter shaft. In cases where the valve was manufactured in its fully crimped diameter, the valve is expanded and then crimped on the balloon. In order to ensure that the balloon reliably expands the frame of the valve after the valve reaches a target implantation site in the patient's vasculature, it may be beneficial to ensure that the crimping process results in the valve being crimped onto the balloon at a repeatably precise location. It also may be beneficial to ensure that the crimping process crimps the frame of the valve evenly and without damage to the valve while ensuring that the crimped diameter of the valve is sufficiently small to traverse the patient's vasculature. The present disclosure addresses these needs.

The present disclosure generally is directed to examples of implantable valve preparations assemblies and to examples of methods of utilizing various components of prosthetic valve preparation assemblies to prepare an implantable prosthetic valve for delivery to a target implantation site.

As shown in FIG. 1, and as discussed in more detail below, a prosthetic valve preparation assembly 10 can comprise an implantable prosthetic valve 300, a valve delivery apparatus 200 configured to deliver the implantable prosthetic valve 300 to the target implantation site, and a crimper assembly 100 for crimping the implantable prosthetic valve around the valve delivery apparatus.

As also shown in FIG. 1, and as discussed in more detail below, the prosthetic valve preparation assembly 10 further can comprise a crimp alignment tool 400 configured to facilitate crimping the implantable prosthetic valve at a target crimping location relative to the valve delivery apparatus 200 and/or a balloon cover assembly 450 configured to selectively engage the valve delivery apparatus 200 and/or the crimp alignment tool 400.

### Exemplary Implantable Prosthetic Valve

Various prosthetic valves are described and/or discussed herein. While specific examples of prosthetic heart valves are discussed herein, general structures, methods of manufacture, and methods of use of various implantable prosthetic valves that can be adapted for use with the apparatuses and methods disclosed herein are described in at least U.S. Pat. Nos. 9,393,110, 10,195,025, 11,013,600, and 11,185,406, the disclosure of each of which is incorporated by reference herein in its entirety.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

FIGS. 1, 11-12, 15, and 17-18 illustrate an example of an implantable prosthetic valve 300. As used herein, the implantable prosthetic valve 300 also may be referred to as a prosthetic valve 300, a valve 300, and/or a transcatheter heart valve (THV) 300. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

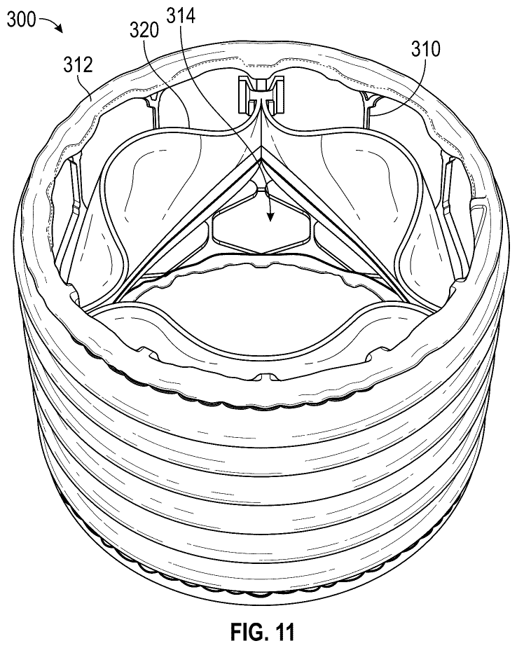

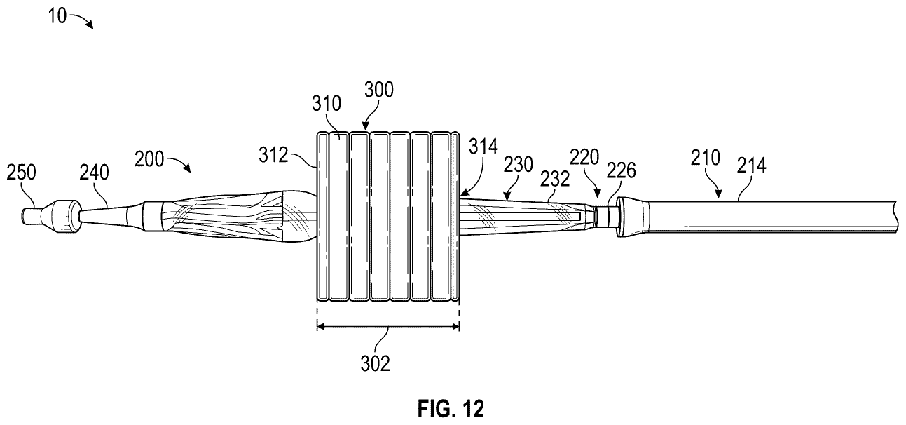

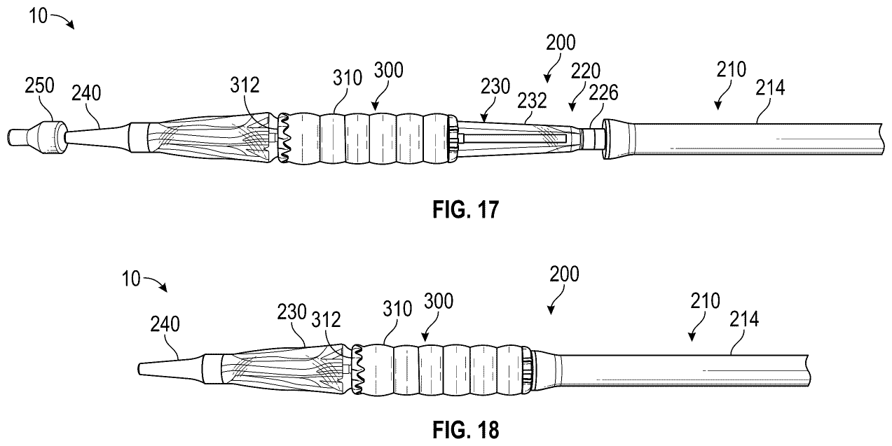

As illustrated in FIG. 11, the implantable prosthetic valve 300 comprises an annular frame 310 defining a frame central passage 314 and a valve assembly 320 positioned within the frame central passage 314. The annular frame 310 is radially expandable and contractable to transition the implantable prosthetic valve 300 between a delivery configuration (see, e.g., FIGS. 17-18) and an operational configuration (see, e.g., FIGS. 11-12). Specifically, when in the delivery configuration, the implantable prosthetic valve 300 is sufficiently reduced in diameter to be sized for delivery through the patient's vasculature. When in the operational configuration, the implantable prosthetic valve 300 is expanded in diameter so as to be operatively mounted at the target implantation site and such that the valve assembly 320 operative to regulate a blood flow within the patient. It is to be understood, however, that the implantable prosthetic valve 300 may be described as being in the operational configuration even when the implantable prosthetic valve 300 is not positioned within the patient and/or at the target implantation site.

The frame 310 can be made of any of various suitable plastically-expandable materials (for example, stainless steel, etc.) or self-expanding materials (for example, Nitinol) as known in the art. When constructed of a plastically-expandable material, the frame 310 (and thus the valve 300) can be crimped to a radially compressed state on a delivery catheter and then expanded inside a patient by an inflatable balloon or equivalent expansion mechanism. When constructed of a self-expandable material, the frame 310 (and thus the valve 300) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 310) include, metal alloys, polymers, or combinations thereof. Example metal alloys can comprise one or more of the following: nickel, cobalt, chromium, molybdenum, titanium, or other biocompatible metal. In some examples, the frame 310 can comprise stainless steel. In some examples, the frame 310 can comprise cobalt-chromium. In some examples, the frame 310 can comprise nickel-cobalt-chromium. In some examples, the frame 310 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

### Examples of a Valve Delivery Apparatus

Described herein are examples of a steerable delivery apparatus (sometimes referred to as a steerable catheter and/or a valve delivery assembly) that can be used to navigate a subject's vasculature to deliver an implantable, expandable medical device (e.g., a prosthetic heart valve), tools, agents, or other therapy to a location within the body of a subject. Examples of procedures in which the steerable catheters are useful include neurological, urological, gynecological, fertility (e.g., in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

Valve delivery assemblies according to examples, and/or components thereof, are shown in FIGS. 1-4 and 12-18. While specific examples of valve delivery assemblies are discussed herein, general structures and methods of use of various valve delivery assemblies that can be adapted for use with the apparatuses and methods disclosed here are described in at least U.S. Pat. Nos. 9,061,119 and 9,339,384, the disclosures of which are incorporated by reference herein in their entirety.

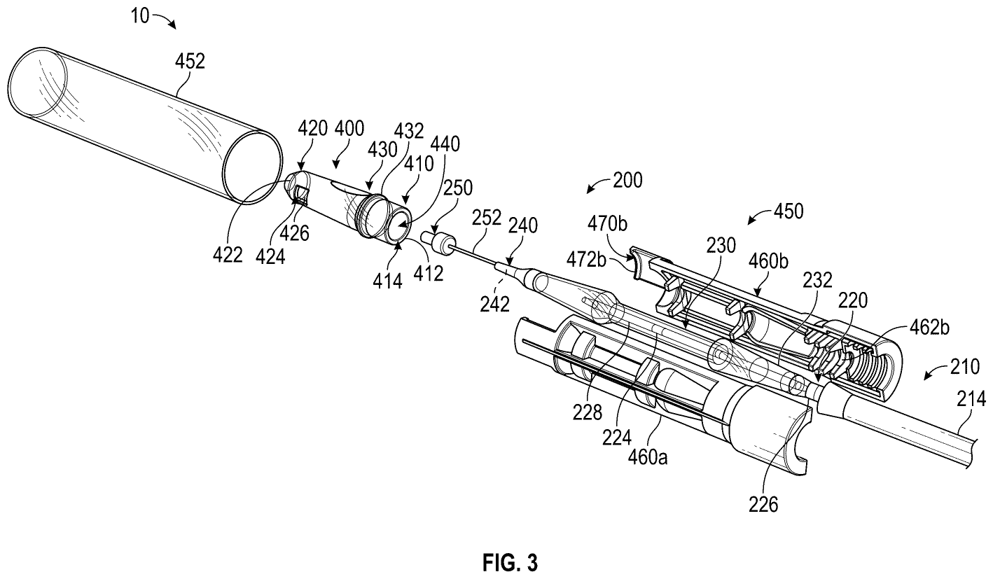

As illustrated at least in FIGS. 1-3, a valve delivery apparatus 200 can comprise a steerable guide catheter 210 and a balloon catheter 220 (shown in FIGS. 1 and 3) extending through the guide catheter 210. As used herein, the guide catheter 210 also may be referred to as a flex catheter 210 and/or a main catheter 210. The use of the term “main catheter” should be understood, however, to include flex or guide catheters, as well as other catheters that do not have the ability to flex or guide through a patient's vasculature.

In some examples, such as in the illustrated example, the guide catheter 210 and the balloon catheter 220 are adapted to slide longitudinally relative to each other to facilitate delivery and positioning of the implantable prosthetic valve 300 at the target implantation site in the patient's body.

As shown in FIG. 1, the guide catheter 210 includes a handle portion 212 and an elongated guide catheter shaft, or tube, 214 extending from the handle portion 212. The balloon catheter 220 includes a proximal portion 222 adjacent to the handle portion 212 and a balloon catheter shaft 226 that extends from the proximal portion 222 and through the handle portion 212 and the guide catheter shaft 214.

In some examples, and as illustrated at least in FIG. 3, the balloon catheter shaft 226 can be an outer balloon catheter shaft 226, and the balloon catheter 220 additionally can comprise an inner balloon catheter shaft 228 that extends from the proximal portion 222 and coaxially through the outer balloon catheter shaft 226 and the inflatable balloon 230. In such examples, the balloon catheter 220 may comprise a fluid passageway that is in fluid communication with an annular space defined between the inner balloon catheter shaft 228 and the outer balloon catheter shaft 226. Specifically, such a fluid passageway can be fluidly connectable to a fluid source for inflating the inflatable balloon, such as to transition the implantable prosthetic valve 300 from the delivery configuration to the operational configuration. In the present disclosure, the operational configuration also may be referred to as a radially expanded configuration of the implantable prosthetic valve 300 and/or of the annular frame 310.

With continued reference to FIGS. 1 and 3, the valve delivery apparatus 200 additionally comprises an inflatable balloon 230 mounted on the balloon catheter 220 at a distal portion 224 of the balloon catheter 220, which is opposite the proximal portion 222. As discussed in more detail herein, the valve delivery apparatus 200 generally is configured such that the implantable prosthetic valve 300 is mounted to the valve delivery apparatus 200 in a crimped state to prepare the valve delivery apparatus 200 and the implantable prosthetic valve 300 for insertion into a patient's vasculature.

In some examples of valve delivery assemblies for transporting implantable prosthetic valves, the valve delivery apparatus is configured such that the implantable prosthetic valve is crimped onto a location that is proximal to, but removed from, the inflatable balloon. Such configurations may facilitate crimping the implantable prosthetic valve to relatively small profile, thereby facilitating delivery of the implantable prosthetic valve through the patient's vasculature. The use of such systems generally necessitates a separate step of repositioning the implantable prosthetic valve upon the inflatable balloon while inside the patient's vasculature.

By contrast, mounting the implantable prosthetic valve directly onto the inflatable balloon prior to entering the patient's vasculature can improve the reliability and ease of implanting the implantable prosthetic valve at the target implantation site. Accordingly, the apparatuses and methods disclosed herein can be used to crimp the implantable prosthetic valve 300 on the inflatable balloon 230 with a tightly compressed profile and at a precise target crimping location relative to the valve delivery apparatus 200.

In some examples, and as illustrated at least in FIG. 3, the valve delivery apparatus 200 comprises a nose piece 240 that is mounted at a distal end of the valve delivery apparatus 200 to facilitate advancement of the valve delivery apparatus 200 through the patient's vasculature to the target implantation site. In some instances, it may be useful to have the nose piece 240 connected to a separate elongated shaft so that the nose piece 240 can move independently of other elements of the valve delivery apparatus 200. The nose piece 240 can be formed of any of a variety of materials, including various polymeric, metallic, and/or composite materials.

In some examples, and as illustrated at least in FIG. 3, the valve delivery apparatus 200 further comprises a stylet 250 that is configured to be removably coupled to a distal end of the nose piece 240. More specifically, and as illustrated in FIG. 3, the nose piece 240 can comprise a nose piece lumen 242, and the stylet 250 can comprise a stylet shaft 252 (e.g., a rigid and/or metallic shaft) that is configured to be received within the nose piece lumen 242 to selectively couple the stylet 250 to the nose piece 240.

In some examples, the stylet 250 is more rigid than the nose piece 240. In particular, the nose piece 240 may be formed of a material that is sufficiently resilient and/or compliant to enable the nose piece 240 to traverse the patient's vasculature without damaging the patient's vasculature. That is, the valve delivery apparatus 200 may be configured such that the nose piece 240 defines a distal end, or terminus, of the valve delivery apparatus 200 when the valve delivery apparatus 200 travels through the patient's vasculature. As described in more detail herein, however, operatively coupling the nose piece 240 to a relatively rigid stylet 250 can facilitate reliably positioning the implantable prosthetic valve 300 at the target crimping location on the inflatable balloon 230 prior to introducing the valve delivery apparatus 200 into the patient's vasculature.

As used herein, the term “operatively coupled,” as used to describe a configuration and/or relationship between two or more components, is intended to refer to a configuration and/or relationship in which the components are directly or indirectly coupled to one another in a manner consistent with the structures and/or functions disclosed herein. For example, a pair of components may be described as being operatively coupled to one another when such components are coupled to one another in a manner that is operative to produce the structural configurations and/or functional properties disclosed herein.

### Examples of Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Examples of a Crimper Assembly

As discussed herein, the prosthetic valve preparation assembly 10 generally comprises a crimper assembly 100 for crimping the implantable prosthetic valve 300 onto the inflatable balloon 230. Crimper assemblies according to some examples, and/or components thereof, are shown in FIGS. 1, 7-10, 13-14, and 16. While specific examples of crimper assemblies are discussed herein, general structures and methods of use of various crimper assemblies that can be adapted for use with the apparatuses and methods disclosed here are described in at least U.S. Pat. No. 7,530,253, the disclosure of which is incorporated by reference herein in its entirety.

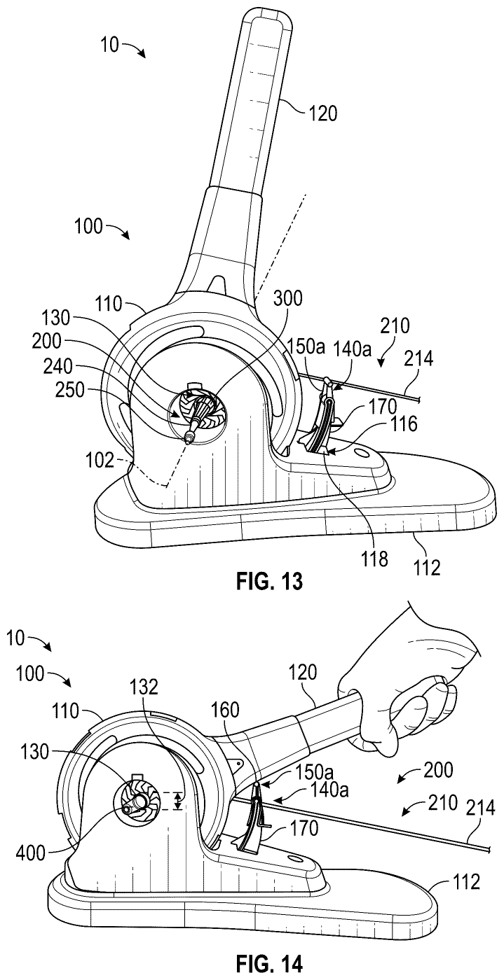

As illustrated at least in FIGS. 1 and 13, a crimper assembly 100 can comprise a base 112, a housing 110 fixedly mounted to the base 112, and a plurality of circumferentially arrayed nesting jaws 114 (labeled in FIG. 1) supported within the housing 110. More specifically, the housing 110 defines a crimper assembly central axis 102, and the jaws 114 are radially moveable within the housing 110 toward and away from the crimper assembly central axis 102. The jaws 114 collectively define a crimper aperture 130 that varies in diameter as the jaws 114 move toward and away from the crimper assembly central axis 102.

The crimper assembly 100 further comprises an actuator handle 120 configured to be manually actuated to move the jaws 114 to vary the diameter of the crimper aperture 130. Specifically, in the illustrated example, the actuator handle 120 can be pivoted relative to the housing 110 in a first direction to move the jaws 114 radially toward the crimper assembly central axis 102, and the actuator handle 120 can be pivoted relative to the housing 110 in a second direction that is opposite the first direction to move the jaws 114 radially away from the crimper assembly central axis 102. Accordingly, when the implantable prosthetic valve 300 is positioned within the crimper aperture 130, actuating the actuator handle 120 to decrease the diameter of the crimper aperture 130 can operate to crimp the implantable prosthetic valve 300.

In some examples, the crimper aperture 130 extends along a crimper aperture length, as measured along a direction parallel to the crimper assembly central axis 102, that is equal to or greater than a valve length 302 of the implantable prosthetic valve 300 (illustrated in FIG. 12). Accordingly, in such examples, the implantable prosthetic valve 300 may be positioned fully within the crimper aperture 130 such that the crimper assembly 100 exerts a crimping force evenly across the full length of the implantable prosthetic valve 300.

When the crimper assembly 100 is utilized to fully crimp the implantable prosthetic valve 300 to the delivery configuration, the implantable prosthetic valve 300 can engage the inflatable balloon 230 sufficiently tightly that the implantable prosthetic valve 300 is restricted from translating relative to the inflatable balloon 230. Thus, prior to fully crimping the implantable prosthetic valve 300 upon the inflatable balloon 230, it is desirable to ensure that the implantable prosthetic valve 300 is properly positioned at the target crimping location.

As used herein, the term “target crimping location” is intended to refer to any suitable axial location and/or range of locations along the inflatable balloon 230 at which the implantable prosthetic valve 300 is to be operatively coupled. As an example, the target crimping location may include and/or be an axial position along the inflatable balloon 230 at which a distal end of the implantable prosthetic valve 300 is to be located when the implantable prosthetic valve 300 is properly crimped to the inflatable balloon 230. As another example, the target crimping location may include and/or be an axial position along the inflatable balloon 230 at which a proximal end of the implantable prosthetic valve 300 is to be located when then implantable prosthetic valve 300 is properly crimped to the inflatable balloon 230. As yet another example, the target crimping location may refer to a portion (e.g., an interval) of the length of the inflatable balloon 230 within which the implantable prosthetic valve 300 is to be positioned when the implantable prosthetic valve 300 is properly crimped to the inflatable balloon 230.

Positioning the implantable prosthetic valve 300 at the target crimping location with a high degree of precision and/or accuracy may facilitate predictable deployment of the implantable prosthetic valve 300 at the target implantation site. In particular, in some examples, positioning the implantable prosthetic valve 300 at the target implantation site is performed by positioning one or more components of the valve delivery apparatus 200 at a known, measurable, and/or controlled location and/or orientation relative to the patient's anatomy. Accordingly, in such examples, controlling a location of the implantable prosthetic valve 300 relative to the delivery apparatus 200 with a high degree of precision and/or accuracy may be beneficial for controlling a location and/or orientation of the implantable prosthetic valve 300 relative to the target implantation site.

Additionally, in some examples, a shape of the implantable prosthetic valve 300 subsequent to being radially expanded by the inflatable balloon 230 is sensitive to a position at which the implantable prosthetic valve 300 is crimped onto the inflatable balloon 230. Accordingly, in such examples, controlling a location of the implantable prosthetic valve 300 relative to the delivery apparatus 200 (e.g., relative to the inflatable balloon 230) with a high degree of precision and/or accuracy may be beneficial for ensuring that the implantable prosthetic valve 300 assumes an intended shape upon being radially expanded by the inflatable balloon 230 to the operational configuration at the target implantation site.

The apparatuses and methods disclosed herein can facilitate and/or enable crimping the implantable prosthetic valve 300 at the target crimping location with any suitable degree of precision and/or accuracy. In particular, in some examples, the apparatuses and methods disclosed herein can consistently yield a crimping accuracy of approximately 1.0-1.5 millimeters (mm) relative to the target crimping location. In some such examples, the target crimping location corresponds to a well-defined location and/or feature of the valve delivery apparatus 200, such as a portion of the nose piece 240 and/or a distal portion of the balloon catheter 220.

When the implantable prosthetic valve 300 is in the operational configuration and positioned near the target crimping location, the diameter of the frame central passage 314 may be sufficiently large that the implantable prosthetic valve 300 engages the valve delivery apparatus 200 only loosely and/or tangentially. Thus, even if the implantable prosthetic valve 300 in the operational configuration is properly positioned at the target crimping location prior to fully crimping the implantable prosthetic valve 300, the crimping action (and/or an axial translation of the valve delivery apparatus 200 during the crimping action) may cause the implantable prosthetic valve 300 to shift to a position away from the target crimping location.

Accordingly, it may be desirable to partially crimp the implantable prosthetic valve 300 to an intermediate configuration prior to fully crimping the implantable prosthetic valve 300 to the delivery configuration. Specifically, in such examples, the implantable prosthetic valve 300 in the intermediate configuration may be sized (e.g., in inner diameter) to engage the valve delivery apparatus 200 and/or the inflatable balloon 230 such that the implantable prosthetic valve 300 may be manually translated relative to the inflatable balloon 230, but such that the implantable prosthetic valve 300 is at least partially restricted from inadvertent translation relative to the inflatable balloon 230. In this manner, crimping the implantable prosthetic valve 300 to the intermediate configuration and subsequently positioning the implantable prosthetic valve 300 at the target crimping location may ensure that the implantable prosthetic valve 300 remains at the target crimping location during the final crimping action.

In some examples, crimping the implantable prosthetic valve 300 to the intermediate configuration or to the delivery configuration is accomplished by bringing the diameter of the crimper aperture 130 to a corresponding predetermined diameter. This in turn can be accomplished by mechanically limiting a range of motion of the actuator handle 120 during use of the crimper assembly 100.

More specifically, and as illustrated in FIGS. 1, 7-10, 13-14, and 16 and as discussed in more detail below, the crimper assembly 100 can comprise a first crimp stop 150 (examples of which are shown in FIGS. 1, 7-8C, 10, and 13-14) and a second crimp stop 170 (shown in FIGS. 1, 7, 9A-10, 13-14, and 16), each of which may be utilized to define a respective range of motion of the actuator handle 120. The second crimp stop 170 can be fixedly coupled relative to the housing 110 during use of the crimper assembly 100, and the first crimp stop 150 can be selectively and operatively coupled to the second crimp stop 170. In particular, and as described in more detail herein, the first crimp stop 150 is configured to be mechanically coupled to the second crimp stop 170.

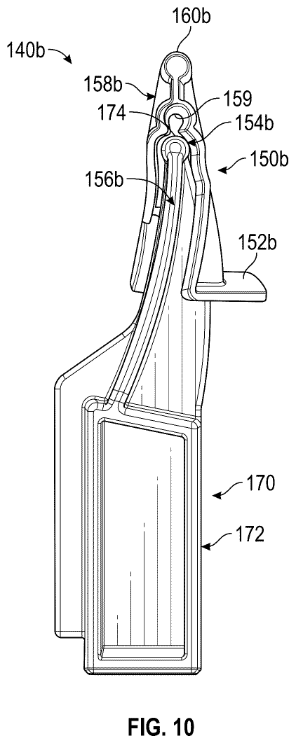

In some examples, the first crimp stop 150 and the second crimp stop 170 may be described as representing components of a crimp stop assembly 140 of the crimper assembly 100. In particular, FIGS. 1, 7-8c, and 13-14 illustrate a first example of a crimp stop assembly 140a that includes a first example of the first crimp stop 150a in combination with the second crimp stop 170, while FIG. 10 illustrates a second example of a crimp stop assembly 140b that includes a second example of the first crimp stop 150b in combination with the second crimp stop 170.

In the present disclosure, and unless otherwise stated (e.g., with reference to a specific illustrated example), references to the crimp stop assembly 140 may be understood as pertaining and/or referring to either or both of the first example crimp stop assembly 140a and/or to the second example crimp stop assembly 140b. Similarly, in the present disclosure, and unless otherwise stated (e.g., with reference to a specific illustrated example), reference to the first crimp stop 150 may be understood as pertaining and/or referring to either or both of the first example first crimp stop 150a and/or to the second example first crimp stop 150b. Accordingly, in the present disclosure, like numerals are utilized to refer to like components of various examples of the crimp stop assembly 140 and/or of the first crimp stop 150, although each such numeral may not be specifically discussed and/or explicitly referenced herein.

When the first crimp stop 150 is operatively coupled to the second crimp stop 170, the first crimp stop 150 and the second crimp stop 170 collectively can define a partial range of motion of the actuator handle 120. That is, when the first crimp stop 150 is operatively coupled to the second crimp stop 170, the actuator handle 120 may be actuated to decrease the diameter of the crimper aperture 130 only until the actuator handle 120 engages the first crimp stop 150, at which point the actuator handle 120 is mechanically restricted and/or prevented from further actuation in this direction.

FIG. 14 illustrates an example in which the implantable prosthetic valve 300 and the valve delivery apparatus 200 (not visible in FIG. 14) are positioned within the crimper aperture 130 and in which the actuator handle 120 is actuated to engage the first crimp stop 150. As shown in FIG. 14, when the actuator handle 120 engages the first crimp stop 150, the crimper aperture 130 may be described as having a first stop diameter 132 that corresponds to the intermediate configuration of the implantable prosthetic valve 300. That is, when the implantable prosthetic valve 300 is positioned within the crimper aperture 130 and the actuator handle 120 is brought into engagement with the first crimp stop 150, the implantable prosthetic valve 300 is crimped to the intermediate configuration as a result of the crimper aperture 130 being brought to the first stop diameter 132. As a result, the outer diameter of the implantable prosthetic valve 300 may be at least substantially equal to the first stop diameter 132 when the implantable prosthetic valve is in the intermediate configuration.

When the first crimp stop 150 is removed from the second crimp stop 170, the second crimp stop 170 can define a full range of motion of the actuator handle 120. That is, when the first crimp stop 150 is removed from the second crimp stop 170 and when the second crimp stop 170 is fixedly coupled relative to the housing 110, the actuator handle 120 may be actuated to decrease the diameter of the crimper aperture 130 only until the actuator handle 120 engages the second crimp stop 170, at which point the actuator handle 120 is mechanically restricted and/or prevented from further actuation in this direction.

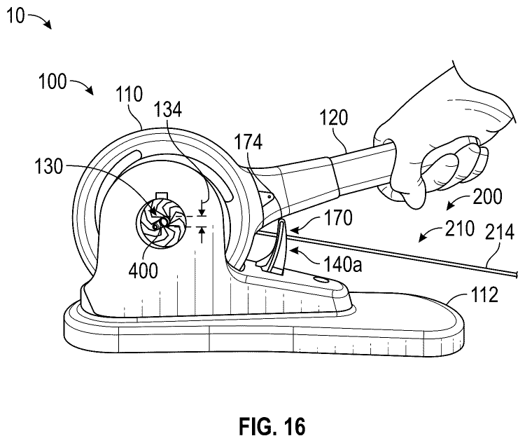

FIG. 16 illustrates an example in which the implantable prosthetic valve 300 and the valve delivery apparatus 200 (not visible in FIG. 16) are positioned within the crimper aperture 130 and in which the actuator handle 120 is actuated to engage the second crimp stop 170. As shown in FIG. 16, when the actuator handle 120 engages the second crimp stop, the crimper aperture 130 may be described as having a second stop diameter 134 that corresponds to the delivery configuration of the implantable prosthetic valve 300 and that is smaller than the first stop diameter 132. That is, when the implantable prosthetic valve 300 is positioned within the crimper aperture 130 and the actuator handle 120 is brought into engagement with the second crimp stop 170, the implantable prosthetic valve 300 is crimped to the delivery configuration as a result of the crimper aperture 130 being brought to the second stop diameter 134. As a result, the outer diameter of the implantable prosthetic valve 300 may be at least substantially equal to the second stop diameter 134 when the implantable prosthetic valve is in the delivery configuration.

In the examples disclosed herein, the first crimp stop 150 may be utilized during an initial step in which the crimper assembly 100 is utilized to crimp the implantable prosthetic valve 300, and/or the second crimp stop 170 may be utilized during a final step in which the crimper assembly 100 is utilized to crimp the implantable prosthetic valve 300. Accordingly, in such examples, the first crimp stop 150 additionally or alternatively may be referred to as an initial crimp stop 150, and/or the second crimp stop 170 additionally or alternatively may be referred to as a final crimp stop 170. Similarly, in such examples, the first stop diameter 132 additionally or alternatively may be referred to as an initial stop diameter 132, and/or the second stop diameter 134 additionally or alternatively may be referred to as a final stop diameter 134. Such examples are not limiting, however, and it additionally is within the scope of the present disclosure that the crimp stop assembly 140 may comprise additional crimp stops, such as a third crimp stop, a fourth crimp stop, etc. In such examples, the plurality of crimp stops (and/or subsets thereof) may be coupled to one another in various combinations to define respective ranges of motion of the actuator handle 120 and/or to produce respective diameters of the crimper aperture 130.

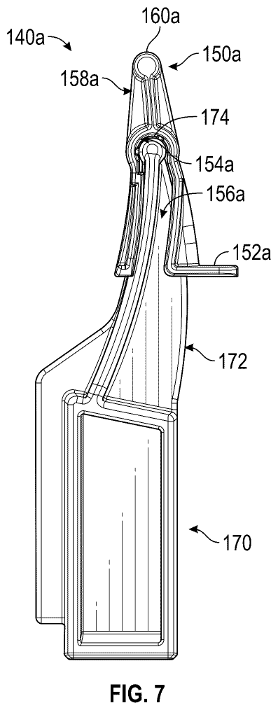

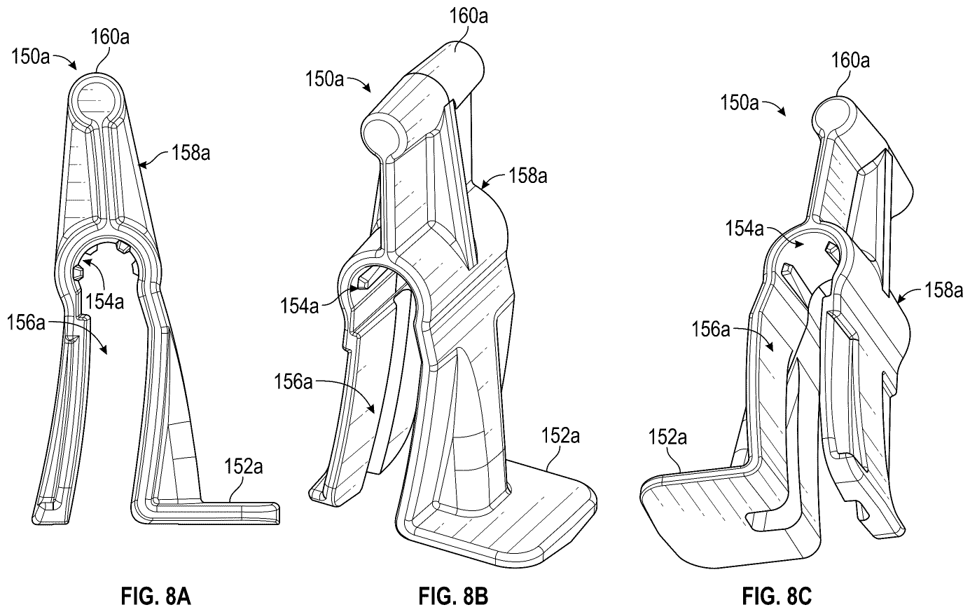

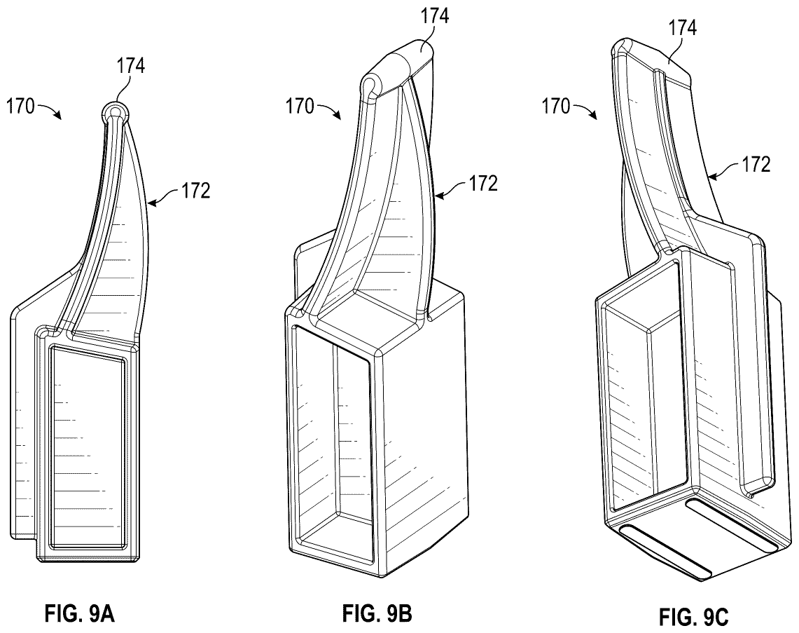

The first crimp stop 150 and the second crimp stop 170 each may have any of a variety of features and/or configurations for exhibiting the functionality disclosed herein. FIGS. 8A-8C illustrate a first example of the first crimp stop 150a, while FIGS. 9A-9C illustrate an example of the second crimp stop 170. FIG. 7 illustrates the first crimp stop 150a of FIGS. 8A-8C operatively coupled to the second crimp stop 170 of FIGS. 9A-9C, while FIG. 10 illustrates another example of the first crimp stop 150b operatively coupled to the second crimp stop 170 of FIGS. 9A-9C.

In some examples, and as shown at least in FIGS. 7-8C, the first crimp stop 150 comprises a first crimp stop body 158 (e.g., the first crimp stop body 158a shown in FIGS. 7-8C) that defines and terminates at a first crimp stop terminal end 160. In such examples, the crimper assembly 100 is configured such that the actuator handle 120 engages the first crimp stop terminal end 160 during use of the crimper assembly 100 to partially crimp the implantable prosthetic valve 300.

Similarly, in some examples, and as shown at least in FIGS. 7 and 9A-9C, the second crimp stop 170 comprises a second crimp stop body 172 that defines and terminates at a second crimp stop terminal end 174. In such examples, the crimper assembly 100 is configured such that the actuator handle 120 engages the second crimp stop terminal end 174 during use of the crimper assembly 100 to fully crimp the implantable prosthetic valve 300.

The first crimp stop 150 and/or the second crimp stop 170 additionally may have any of a variety of features to facilitate operatively coupling the first crimp stop 150 to the second crimp stop 170. For example, in some examples, and as shown in FIGS. 7-8C, the first crimp stop 150 comprises a second crimp stop receiver 154 (e.g., the second crimp stop receiver 154a shown in FIGS. 7-8C) that is configured to receive a portion of the second crimp stop 170, such as the second crimp stop body 172 and/or the second crimp stop terminal end 174, when the first crimp stop 150 is operatively coupled to the second crimp stop 170. As a more specific example, the second crimp stop receiver 154 may comprise and/or be a receiver channel 156 (e.g., the receiver channel 156a shown in FIGS. 7-8C) that receives an elongate portion of the second crimp stop 170 to mechanically couple the first crimp stop 150 to the second crimp stop 170.

In some examples, the internal geometry of the first crimp stop 150 (e.g., the receiver channel 156) is configured to receive the top portion of the second crimp stop 170 (e.g., a portion including the second crimp stop terminal end 174) in a mating configuration. For example, the receiver channel 156 may be shaped similar to the top portion of the second crimp stop 170 such that the first crimp stop 150 may be snapped onto the second crimp stop 170. Such mechanical engagement also may serve to prevent the first crimp stop 150 from being inadvertently removed from the second crimp stop 170.

Additionally or alternatively, in some examples, such as in the example of FIGS. 7-8C, the first crimp stop 150 comprises a grip feature 152 (e.g., the grip feature 152a shown in FIGS. 7-8C) configured to facilitate removal of the first crimp stop 150 from the second crimp stop 170. In particular, when present, the grip feature 152 can be configured to be gripped by a user to exert a torque on the first crimp stop 150 and/or to enable the user to pull the first crimp stop 150 away from the second crimp stop 170. The grip feature 152 may include and/or be any of a variety of structures, examples of which include a tab, a lever, a protrusion, and indentation, etc. In some examples, the grip feature 152 is at least partially defined by the first crimp stop body 158.

The second example of the first crimp stop 150b shown in FIG. 10 is substantially similar to the first example of the first crimp stop 150a shown in FIGS. 7-8C, with the exception that the first crimp stop body 158b additionally defines an upper recess 159 that is connected to (e.g., open to) the receiver channel 156b. In particular, and as shown in FIG. 10, when the first crimp stop 150b is coupled to the second crimp stop 170, the second crimp stop body 172 occupies the receiver channel 156b but does not occupy the upper recess 159, which in turn is positioned between the first crimp stop terminal end 160b and the second crimp stop terminal end 174. Such a configuration may facilitate flexure of the first crimp stop body 158b, such as when pulling and/or applying a torque to the grip feature 152b to remove the first crimp stop 150b from the second crimp stop 170.

As discussed above, the crimper assembly 100 generally is configured such that the second crimp stop 170 is fixedly coupled relative to the housing 110 during use of the crimper assembly 100. In particular, as shown in FIG. 13, during use of the crimper assembly 100, the second crimp stop 170 can be coupled to the base 112 and/or to the housing 110 at a crimp stop attachment location 118 that is defined by the base 112 and/or the housing 110.

In some examples, the base 112 and/or the housing 110 can comprise a crimp stop receiver 116 that defines the crimp stop attachment location 118. In such examples, the crimp stop receiver 116 may comprise and/or be any suitable structure and/or mechanism for fixedly retaining the second crimp stop 170, examples of which include a friction-fit coupling, a snap-fit coupling, a latching coupling, a keyed coupling, etc.

In some examples, the second crimp stop 170 may be permanently or semi-permanently attached to the crimp stop attachment location 118. Stated differently, in such examples, the second crimp stop 170 may not be configured to be removed from the crimp stop attachment location 118 without damage to the crimper assembly 100. This is not required of all examples of the crimper assembly 100, however, and it additionally is within the scope of the present disclosure that the second crimp stop 170 may be configured to be selectively and/or temporarily attached to the crimp stop attachment location 118. Stated differently, in some examples, the second crimp stop 170 may be configured to be selectively and repeatedly coupled to and removed from the crimp stop attachment location 118 without damage to the crimper assembly 100.

### Examples of a Crimp Alignment Tool

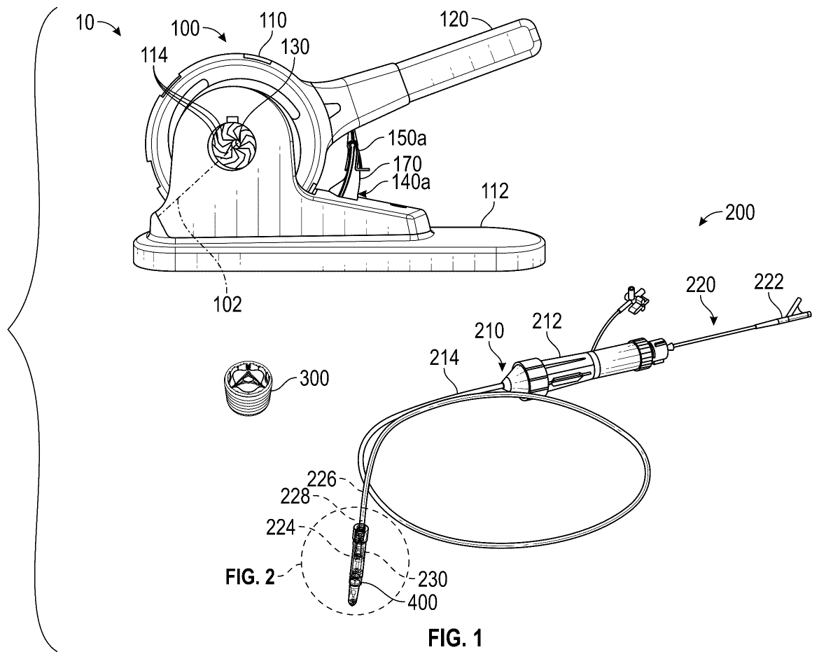

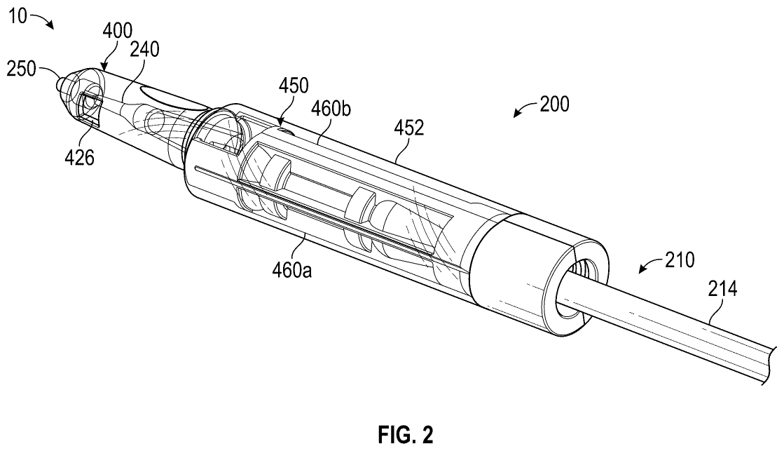

As discussed above, it generally is desirable to ensure that the implantable prosthetic valve 300 is precisely and reliably positioned at the target crimpling location relative to the inflatable balloon 230 prior to fully crimping the implantable prosthetic valve 300 onto the inflatable balloon 230. Accordingly, and as shown at least in FIGS. 2-4, 6A-6D, and 15, the prosthetic valve preparation assembly 10 can comprise a crimp alignment tool 400 that is configured to facilitate positioning the implantable prosthetic valve 300 at the target crimping location.

The crimp alignment tool 400 can be utilized to ensure that the implantable prosthetic valve 300 is positioned at a predetermined axial position relative to one or more components of the valve delivery apparatus 200 that corresponds to the target crimping location. Specifically, and as discussed in more detail herein, the crimp alignment tool 400 can be configured to engage each of the valve delivery apparatus 200 and the implantable prosthetic valve 300 to position the implantable prosthetic valve 300 at the target crimping location relative to the valve delivery apparatus 200.

As discussed above, when the implantable prosthetic valve 300 is in the intermediate configuration (e.g., subsequent to being partially crimped by the crimper assembly 100), the implantable prosthetic valve 300 may be manually translated along a length of the valve delivery apparatus 200 and/or of the inflatable balloon 230, but may otherwise be restricted from inadvertent translation relative to the valve delivery apparatus 200. Accordingly, in some examples, the crimp alignment tool 400 is utilized to position the implantable prosthetic valve 300 while the implantable prosthetic valve 300 is in the intermediate configuration.

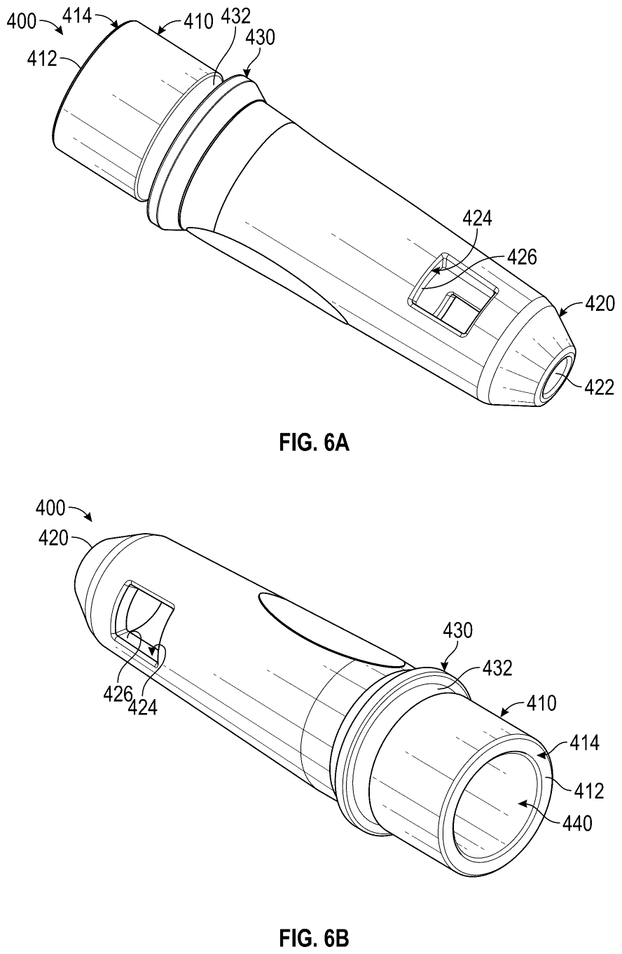

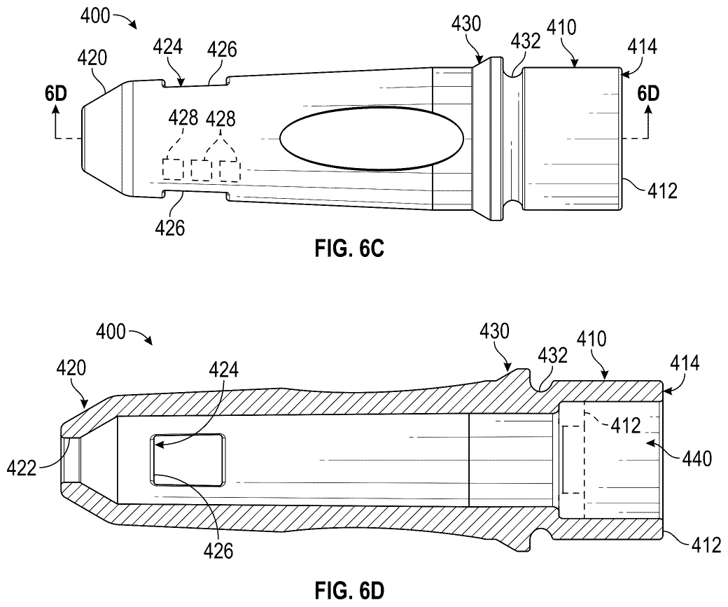

The crimp alignment tool 400 may have any of a variety of characteristics and/or features for engaging the valve delivery apparatus 200 and/or the implantable prosthetic valve 300. In some examples, and as shown at least in FIGS. 3, 6B, and 6D, the crimp alignment tool 400 comprises a catheter receiver 440 configured to receive a distal portion of the valve delivery apparatus 200. In particular, the crimp alignment tool 400 is configured such that, when the distal portion of the valve delivery apparatus 200 is operatively received within the catheter receiver 440 (as described herein), the crimp alignment tool 400 may be described as being in an alignment configuration relative to the valve delivery apparatus 200.

The alignment configuration of the crimp alignment tool 400 may be described and/or defined with reference to a relative orientation and/or a mechanical engagement between the crimp alignment tool 400 and the valve delivery apparatus 200. In particular, and as described in more detail herein, the crimp alignment tool 400 can comprise a crimp alignment tool distal end 420 that is configured to engage the distal portion of the valve delivery apparatus 200 to define the alignment configuration and/or to retain the crimp alignment tool 400 in the alignment configuration.

When the crimp alignment tool 400 is in the alignment configuration relative to the valve delivery apparatus 200, the position of the crimp alignment tool 400 may be utilized to place the implantable prosthetic valve 300 at the target crimping location. In particular, and as illustrated in FIGS. 3, 6A-6D, and 15, the crimp alignment tool 400 comprises a valve stop 412 positioned in a proximal region 410 of the crimp alignment tool 400.

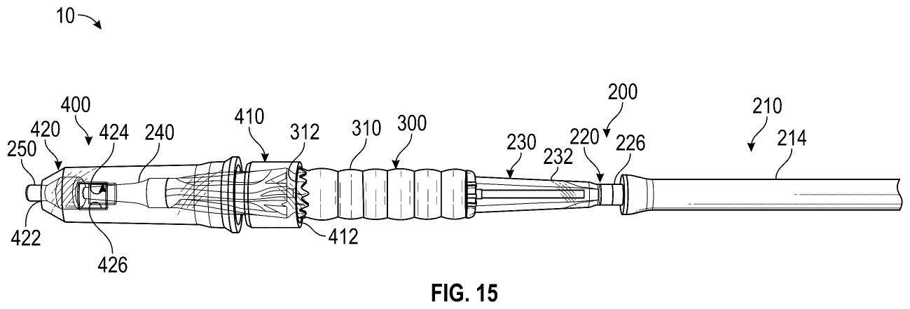

As shown in FIG. 15, the valve stop 412 is configured to engage a distal frame end 312 of the annular frame 310 of the implantable prosthetic valve 300. Specifically, the crimp alignment tool 400 is configured such that, when the crimp alignment tool 400 is in the alignment configuration with the crimp alignment tool distal end 420 engaging the distal portion of the valve delivery apparatus 200, positioning the implantable prosthetic valve 300 such that the distal frame end 312 engages the valve stop 412 operates to position the implantable prosthetic valve 300 at the target crimping location. As discussed in more detail below, the valve stop 412 may comprise and/or be a structure that is defined by the crimp alignment tool 400, such as an annular surface and/or terminal edge thereof.

The crimp alignment tool 400 may be configured to engage the valve delivery apparatus 200 in the alignment configuration in any of a variety of manners. In some examples, the crimp alignment tool 400 can be moved longitudinally (e.g., via axial translation) onto the distal portion of the valve delivery apparatus 200 to operatively couple the crimp alignment tool 400 to the valve delivery apparatus 200. Similarly, in such examples, the crimp alignment tool 400 may be removed from the valve delivery apparatus 200 by longitudinally sliding the crimp alignment tool 400 off of and away from the valve delivery apparatus 200.

In some examples, the crimp alignment tool 400 can be configured to restrict and/or prevent inadvertent removal of the crimp alignment tool 400 from the valve delivery apparatus 200. For example, the crimp alignment tool 400 can be configured such that, when the crimp alignment tool 400 is in the alignment configuration, the crimp alignment tool 400 is at least partially restricted from being removed from the valve delivery apparatus 200. As more specific examples, the crimp alignment tool 400 may be configured to engage the distal portion of the valve delivery apparatus in a friction-fit engagement, a mechanical engagement, a latching engagement, a snap-fit engagement, etc.

In some examples, positioning the crimp alignment tool 400 in the alignment configuration may produce a tactile and/or auditory confirmation, such as a snap, a pop, a bump, etc., that indicates that the crimp alignment tool has reached the alignment configuration. Such a tactile and/or auditory confirmation can be the result, for example, of forming a mechanical coupling, such as engaging a latch, engaging/receiving a detent, engaging/receiving a projection or a groove, etc.

The crimp alignment tool 400 may be configured to receive and/or engage any suitable portion of the valve delivery apparatus 200 for defining, and/or for retaining the crimp alignment tool 400 in, the alignment configuration. In some examples, and as illustrated in FIGS. 2, 4, and 15, the crimp alignment tool 400 is configured such that the stylet 250 of the valve delivery apparatus 200 engages the crimp alignment tool distal end 420 when the crimp alignment tool 400 is in the alignment configuration. In such examples, the crimp alignment tool 400 can be configured such that an inner diameter of the crimp alignment tool 400 mates with an outer diameter of the stylet 250 when the crimp alignment tool 400 is in the alignment configuration.

In particular, and as discussed above, the stylet 250 may be formed of a material that is more rigid than the nose piece 240, and thus may provide a reliable point of reference for measuring an axial distance relative to the inflatable balloon 230. For example, when the stylet 250 is formed of a relatively rigid material, such rigidity may facilitate establishing a precise and/or well-defined positional relationship between the stylet 250 and the crimp alignment tool 400.

More specifically, in some examples, the stylet shaft 252 extend within the nose piece lumen 242 to engage a relatively rigid component of the valve delivery apparatus 200, such as a distal end of the balloon catheter 220. Thus, in such examples, when the stylet 250 is operatively coupled to the nose piece 240, an axial force applied to the stylet 250 may be communicated via the stylet shaft 252 to the balloon catheter 220. Because the axial position of the distal end of the balloon catheter 220 is substantially fixed relative to the inflatable balloon 230 (and thus relative to the target crimping location), the stylet 250 itself thus may serve as reference location for locating the target crimping location. That is, the stylet 250 and/or the stylet shaft 252 may be sized such that, when the stylet shaft 252 engages the distal end of the balloon catheter 220 via the nose piece lumen 242 and when the crimp alignment tool 400 receives and engages the stylet 250, the valve stop 412 is aligned with the target crimping location (and/or the distal end thereof). In this manner, such a configuration may facilitate establishing a precise and substantially constant axial positional relationship between the crimp alignment tool 400 and the target crimping location, thus ensuring that the crimp alignment tool 400 may be utilized to properly position the implantable prosthetic valve 300.

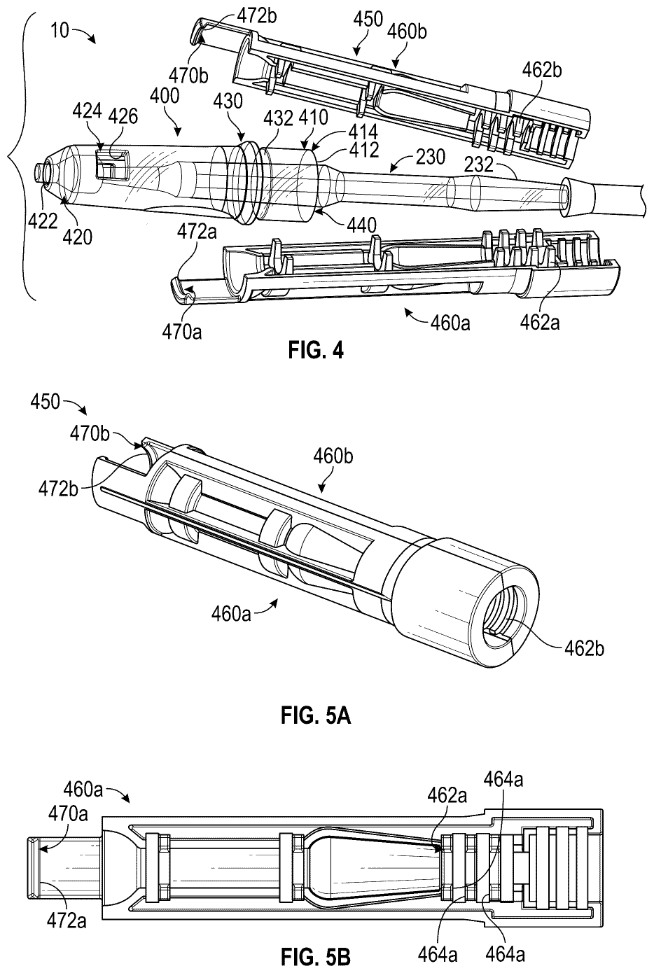

In some examples, and as illustrated at least in FIG. 4, the crimp alignment tool distal end 420 comprises a distal aperture 422 that receives the distal portion of the valve delivery apparatus 200 (e.g., the stylet 250) when the crimp alignment tool is in the alignment configuration.

In particular, in some such examples, the crimp alignment tool 400 can be configured such that the stylet 250 extends at least partially through the distal aperture 422 when the crimp alignment tool 400 is in the alignment configuration. In this manner, the engagement between the distal aperture 422 and the stylet 250 may at least partially define the alignment configuration.

For example, the stylet 250 can be at least partially conical in shape such that the stylet 250 engages the distal aperture 422 (i.e., the portion of the crimp alignment tool 400 that defines the distal aperture 422) only when the stylet 250 is in a precise and predetermined axial position relative to the crimp alignment tool 400.

In some examples, the crimp alignment tool 400 can comprise a distal alignment indicator 424 in the form of one or more features for providing a visual verification that the crimp alignment tool 400 is in the alignment configuration. For example, in some examples, and as illustrated in FIGS. 2-4, 6A-6D, and 15, the distal alignment indicator 424 comprises a window 426 that permits visual inspection of the valve delivery apparatus 200 through the crimp alignment tool 400. That is, in such examples, the window 426 can be an opening and/or an aperture formed in a side wall of the crimp alignment tool 400 such that a portion of the valve delivery apparatus 200 that is received within the crimp alignment tool 400 is visible through the window 426.

In some examples, the crimp alignment tool 400 is configured such that, when the crimp alignment tool is in the alignment configuration, a component of the valve delivery apparatus is axially aligned with a distal edge of the window 426 and/or with a proximal edge of the window 426. In particular, in some examples, and illustrated at least in FIG. 15, the window 426 is positioned such that a proximal edge of the stylet 250 is axially aligned with a distal edge of the window 426 when the crimp alignment tool 400 is in the alignment configuration. In this manner, the user can verify that the crimp alignment tool 400 is in the alignment configuration by visually verifying that the proximal edge of the stylet 250 is axially aligned with the distal edge of the window 426.

Additionally or alternatively, in some examples, and as schematically illustrated in FIG. 6C, the distal alignment indicator 424 can comprise one or more visual indicia 428 that provides another visual indicator that the crimp alignment tool 400 is in the alignment configuration. For example, the visual indicia 428 can be configured such that a component of the valve delivery apparatus 200 (e.g., the nose piece 240 and/or the stylet 250) is axially aligned with the visual indicia 428 when the crimp alignment tool 400 is in the alignment configuration. As examples, the visual indicia 428 can comprise and/or be a mark, a colored mark, a printed mark, an embossed mark, a debossed mark, a line, a symbol, etc.

The crimp alignment tool 400 may be configured to engage the implantable prosthetic valve 300 to position the implantable prosthetic valve 300 at the target crimping location in any suitable manner. In some examples, and as illustrated at least in FIGS. 6A-6C, the crimp alignment tool 400 comprises and/or terminates at a crimp alignment tool proximal end 414 that defines the valve stop 412. Stated differently, in such examples, positioning the implantable prosthetic valve 300 such that the distal frame end 312 engages and/or abuts the crimp alignment tool proximal end 414 while the crimp alignment tool 400 is in the alignment configuration operates to position the implantable prosthetic valve at the target crimping location.

This is not required of all examples of the crimp alignment tool, however. For example, it additionally is within the scope of the present disclosure that the crimp alignment tool 400 is configured to at least partially axially overlap with the implantable prosthetic valve 300 to position the implantable prosthetic valve 300 at the target crimping location. As a more specific example, and as schematically illustrated in FIG. 6D, the crimp alignment tool 400 can comprise a stepped interior surface that defines the valve stop 412 in the form of an annular shoulder or protrusion, such that the implantable prosthetic valve 300 is partially received within the crimp alignment tool 400 when the distal frame end 312 engages the valve stop 412.

### Examples of a Balloon Cover Assembly

In some example, the prosthetic valve preparation assembly 10 additionally may comprise one or more features for preserving a shape and/or configuration of the valve delivery apparatus 200 and/or the crimp alignment tool 400 prior to crimping the implantable prosthetic valve 300 onto the inflatable balloon 230. For example, in some examples, and as illustrated in FIGS. 2-5D, the prosthetic valve preparation assembly 10 comprises a balloon cover assembly 450 that is configured to be operatively coupled to the inflatable balloon 230, such as to shape and/or protect the inflatable balloon 230 prior to preparing the valve delivery apparatus 200 to be deployed into a patient's vasculature. More specifically, and as described in more detail herein, the balloon cover assembly 450 may be configured to cover and/or engage the inflatable balloon 230, such as to protect the inflatable balloon 230 from damage or misshaping while in transit to the user.

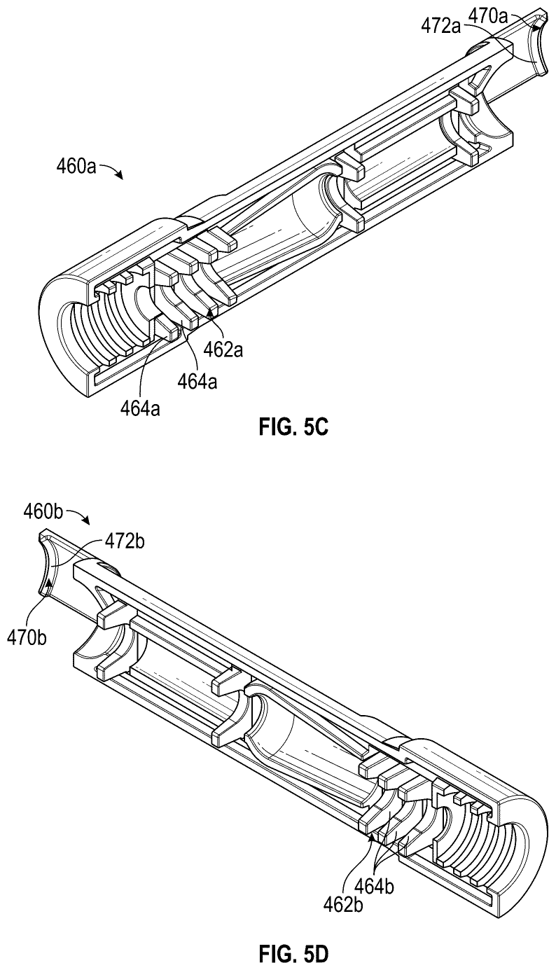

In some examples, and as illustrated in FIGS. 2-5D, the balloon cover assembly 450 can comprise a pair of balloon cover clamshell portions 460a and 460b that are configured to be mounted to opposite sides of the inflatable balloon 230 when the balloon cover assembly 450 is operatively coupled to the inflatable balloon 230. In particular, FIG. 2 illustrates the balloon cover clamshell portions 460a and 460b operatively coupled to the inflatable balloon 230, while FIGS. 3-4 illustrate the balloon cover clamshell portions 460a and 460b removed from the inflatable balloon 230. As discussed below, FIGS. 5A-5C illustrate the balloon cover clamshell portions 460a and/or 460b in more detail. In the present disclosure, the balloon cover clamshell portion 460a also may be referred to as a first balloon cover clamshell portion 460a, and/or the balloon cover clamshell portion 460b also may be referred to as a second balloon cover clamshell portion 460b.

The balloon cover clamshell portions 460a and 460b collectively can engage the inflatable balloon 230, such as to maintain the balloon cover assembly 450 in position relative to the inflatable balloon 230 and/or to preserve a shape of the inflatable balloon 230. As used herein, each of the balloon cover clamshell portions 460a and 460b may be described as representing a respective example of a balloon cover clamshell portion 460.

In some examples, the balloon cover assembly 450 additionally comprises one or more features to restrict the balloon cover clamshell portions 460a and 460b from being removed from the inflatable balloon 230. In particular, in some examples, and as illustrated in FIGS. 2-3, the balloon cover assembly 450 comprises a balloon cover sleeve 452 that is configured to surround the balloon cover clamshell portions 460a and 460b and to retain the balloon cover clamshell portions 460a and 460b relative to the inflatable balloon 230. Stated differently, when the balloon cover assembly 450 is operatively coupled to the inflatable balloon 230 and when the balloon cover clamshell portions 460a and 460b are received within the balloon cover sleeve 452, the balloon cover sleeve 452 can restrict and/or prevent the balloon cover clamshell portions 460a and 460b from being removed from the inflatable balloon 230 until the balloon cover sleeve 452 is removed from the balloon cover clamshell portion 460a and 460b.

In some examples, and with particular reference to FIGS. 2-3 and 5A, the balloon cover clamshell portions 460a and 460b collectively form a cylindrical outer surface when operatively coupled to the inflatable balloon 230, and the balloon cover sleeve 452 (shown in FIG. 3) comprises a cylindrical inner surface that receives and engages the balloon cover clamshell portions 460a and 460b. In this manner, in this example, the balloon cover sleeve 452 may be installed onto and removed from the balloon cover clamshell portions 460a and 460b by longitudinally sliding the balloon cover sleeve 452 onto and off of the balloon cover clamshell portions 460a and 460b.

Such a configuration is not required, however, and it additionally is within the scope of the present disclosure that the balloon cover sleeve 452 may have any suitable form for engaging the balloon cover clamshell portions 460a and 460b. For example, the balloon cover clamshell portions 460a and 460b and the balloon cover sleeve 452 can comprise respective mating surfaces that engage one another (e.g., via a frictional interface and/or a mechanical coupling), such as to restrict the balloon cover sleeve 452 from being inadvertently removed from the balloon cover clamshell portions 460a and 460b.

Each of the balloon cover clamshell portion 460a and 460b may be configured to engage the inflatable balloon 230 in any of a variety of manners. In some examples, and as illustrated at least in FIGS. 5C-5D, each of the balloon cover clamshell portions 460a and/or 460b can comprise a respective proximal balloon engagement surface 462a or 462b (respectively) that engages the inflatable balloon 230 when the balloon cover assembly 450 is operatively coupled to the inflatable balloon 230. In particular, the engagement between the proximal balloon engagement surfaces 462 and the inflatable balloon 230 can facilitate establishing and/or preserving a formed geometry of the inflatable balloon 230. Additionally or alternatively, the engagement between the proximal balloon engagement surfaces 462a and 462b and the inflatable balloon 230 can restrict the balloon cover clamshell portions 460 from translating axially relative to the inflatable balloon 230 when the balloon cover assembly 450 is operatively coupled to the inflatable balloon 230.

Each of the proximal balloon engagement surface 462a and 462b may include and/or be any of a variety of structural features for engaging the inflatable balloon 230. For example, and as illustrated at least in FIGS. 5C-5D, each of the proximal balloon engagement surfaces 462a and/or 462b may comprise and/or be one or more locking ribs 464 that extend radially toward and/or engage the inflatable balloon 230 when the balloon cover assembly 450 is operatively coupled to the inflatable balloon 230. More specifically, in the illustrated example, the proximal balloon engagement surface 462a comprises four locking ribs 464a, while the proximal balloon engagement surface 462b comprises three locking ribs 464b that are staggered relative to the locking ribs 464a. Thus, when the balloon cover clamshell portions 460a and 460b are assembled to one another, the locking ribs 464a and 464b are interleaved with one another.

In some examples, the proximal balloon engagement surfaces 462a and 462b may be configured to engage one another when the balloon cover assembly 450 is operatively coupled to the inflatable balloon 230. As a more specific example, when each of the proximal balloon engagement surfaces 462a and 462b comprise respective locking ribs 464a and 464b, the locking ribs 464a may be configured to be interleaved with and to frictionally engage the locking ribs 464b, thus restricting the balloon cover clamshell portions 460a and 460b from being removed from one another. Stated differently, in such an example, the proximal balloon engagement surfaces 462a and 462b are configured to engage one another to mechanically retain the balloon cover clamshell portions 460a and 460b against one another when the balloon cover assembly 450 is operatively coupled to the inflatable balloon 230.

As discussed above, the balloon cover clamshell portions 460a/460b and/or the proximal balloon engagement surfaces 462a/462b thereof may engage the inflatable balloon 230 to facilitate establishing and/or preserving a geometry of the inflatable balloon 230. In particular, the engagement between the proximal balloon engagement surfaces 462a/462b and the inflatable balloon 230 may operate to slightly compress and/or to mechanically shape the inflatable balloon 230 (such as a proximal portion 232 of the inflatable balloon 230, as shown in FIGS. 3-4).

Additionally or alternatively, such engagement may operate to form the inflatable balloon 230 into a profile of decreased radius (e.g., relative to a profile prior to operatively coupling the balloon cover assembly 450 to the inflatable balloon 230) that may persist upon removal of the balloon cover clamshell portions 460a/460b from the inflatable balloon 230. Such a constricted profile may facilitate ensuring that the final profile of the inflatable balloon 230 (e.g., after utilizing the crimper assembly 100 as disclosed herein) is sufficiently small to extend within the inner diameter of the guide catheter shaft 214.

In some examples, the pair of balloon cover clamshell portions 460a and 460b are at least substantially similar and/or identical to one another. For example, in the example of FIGS. 5A-5D, the balloon cover clamshell portions 460a and 460b are identical to one another aside from the configuration and number of locking ribs 464 of each respective proximal balloon engagement surface 462a/462b. Specifically, the balloon cover clamshell portion 460a (FIG. 5C) includes a proximal balloon engagement surface 462a with four locking ribs 464, while the balloon cover clamshell portion 460b (FIG. 5D) includes a proximal balloon engagement surface 462b with three locking ribs 464.

While the present disclosure generally relates to examples in which the balloon cover assembly 450 comprises a pair of balloon cover clamshell portions 460 (e.g., the balloon cover clamshell portions 460a and 460b), this is not required of all examples of the balloon cover assembly 450. For example, it additionally is within the scope of the present disclosure that the balloon cover assembly 450 may comprise any suitable number of balloon cover clamshell portions 460 for engaging the inflatable balloon 230 as described herein, such as three balloon cover clamshell portions 460, four balloon cover clamshell portions 460, or more than four balloon cover clamshell portions 460.

In some examples, and as illustrated at least in FIGS. 2 and 5A, the pair of balloon cover clamshell portions 460 are configured to engage one another when the balloon cover assembly is operatively coupled to the inflatable balloon 230. More specifically, the pair of balloon cover clamshell portions 460 may be configured to abut one another and/or to mate with one another.

This is not required of all examples of the balloon cover assembly 450, however, and it additionally is within the scope of the present disclosure that the balloon cover clamshell portions 460 may be spaced apart from one another when the balloon cover assembly 450 is operatively coupled to the inflatable balloon 230.

In some examples, the balloon cover assembly 450 additionally or alternatively may be configured to engage the crimp alignment tool 400 when the balloon cover assembly 450 is operatively coupled to the inflatable balloon 230. For example, the balloon cover assembly 450 may be configured to engage the crimp alignment tool 400 such that the balloon cover assembly 450 restricts the crimp alignment tool 400 from being removed from the valve delivery apparatus 200 when the balloon cover assembly 450 is operatively coupled to the inflatable balloon 230 and when the crimp alignment tool 400 is in the alignment configuration.

As a more specific example, and illustrated at least in FIG. 3, the crimp alignment tool 400 can comprise a clamshell engagement feature 430, and each balloon cover clamshell portion 460 can comprise a crimp alignment tool engagement feature 470 that engages the clamshell engagement feature 430 when the balloon cover assembly 450 operatively engages the crimp alignment tool 400.

The clamshell engagement feature 430 and the crimp alignment tool engagement features 470 may comprise and/or be any suitable features, such as features that mate with one another and/or mechanically engage one another to restrict and/or prevent axial translation of the crimp alignment tool 400 relative to the balloon cover assembly 450. As a more specific example, and as illustrated in FIG. 3, the clamshell engagement feature 430 can comprise an annular groove 432, and each crimp alignment tool engagement feature 470 can comprise an engagement tooth 472. In particular, FIG. 3 labels the engagement tooth 472b of the second balloon cover clamshell portion 460b, while the engagement tooth 472a of the first balloon cover clamshell portion 460a is more clearly illustrated in FIG. 4.

As illustrated in FIG. 4, each engagement tooth 472a and 472b may extend radially inwardly such that each engagement tooth extends within the annular groove 432 when the balloon cover assembly 450 operatively engages the crimp alignment tool 400. Accordingly, in such examples, when the balloon cover assembly 450 operatively engages the crimp alignment tool 400, each engagement tooth 472 is received within a portion of the annular groove 432 to restrict the crimp alignment tool 400 from translating axially relative to the balloon cover assembly 450.

In this manner, in the illustrated example, each crimp alignment tool engagement feature 470 may be described as a male mating feature, and the clamshell engagement feature 430 may be described as a female mating feature. This is, however, not required of all examples of the balloon cover assembly and/or of the crimp alignment tool. For example, it also is within the scope of the present disclosure that each crimp alignment tool engagement feature 470 additionally or alternatively may include and/or be a female mating feature (e.g., a groove, a channel, a trough, a recess, an indentation, etc.). Similarly, it also is within the scope of the present disclosure that the clamshell engagement feature 430 additionally or alternatively may include and/or be a male mating feature (e.g., a tooth, a protrusion, a ridge, etc.).

### Exemplary Methods

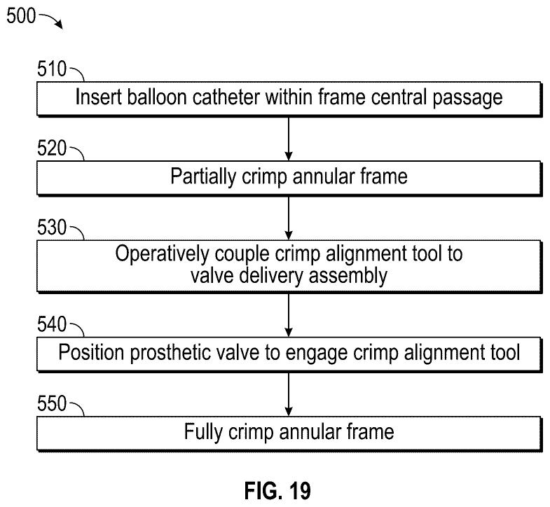

Turning now to FIG. 19, the present disclosure provides for methods of mounting an implantable prosthetic valve to a valve delivery apparatus for delivering the implantable prosthetic valve to a target implantation site within a patient's vasculature. The methods can be performed in conjunction with any suitable components of the prosthetic valve preparation assemblies 10 disclosed herein. Accordingly, in the present disclosure, any description of a component, a feature, a characteristic, a configuration, etc. that is presented with reference to a step of a method may be understood as referring to any corresponding (e.g., similarly named) components, features, characteristics, configurations, etc. disclosed herein with reference to the prosthetic valve preparation assembly 10 and/or with reference to FIGS. 1-18.

As shown in FIG. 19, the method 500 can comprise, at 510, inserting a balloon catheter of the valve delivery apparatus within a frame central passage of the implantable prosthetic valve. For example, the inserting the balloon catheter within the frame central passage at 510 may comprise longitudinally sliding the implantable prosthetic valve onto the distal portion of the valve delivery apparatus, such as to position the implantable prosthetic valve around and/or proximate to the inflatable balloon.

FIG. 12 may be described as illustrating an example of a configuration of the implantable prosthetic valve and the valve delivery apparatus subsequent to performing the inserting the balloon catheter within the frame central passage at 510.

In some examples, and as discussed above, the valve delivery apparatus can comprise a balloon cover assembly with a pair of balloon cover clamshell portions and a balloon cover sleeve. In some such examples, the method 500 may be initiated and/or at least partially performed while the balloon cover assembly is operatively coupled to the inflatable balloon. Accordingly, in some examples, the method 500 additionally comprises, prior to the inserting the balloon catheter within the frame central passage at 510, removing the balloon cover assembly from the inflatable balloon.

In some such examples, the removing the balloon cover assembly comprises removing the balloon cover sleeve from the pair of balloon cover clamshell portions (e.g., by sliding the balloon cover sleeve axially off of and away from the balloon cover clamshell portions). Subsequent to removing the balloon cover assembly, the method can comprise removing the pair of balloon cover clamshell portions from the inflatable balloon.

At 520, the method 500 can comprise partially crimping an annular frame of the implantable prosthetic valve with a crimper assembly. As discussed herein, the crimper assembly may be utilized to partially crimp the annular frame of the implantable prosthetic valve to bring the implantable prosthetic valve to an intermediate configuration, in which the implantable prosthetic valve may be selectively but deliberately positioned along the valve delivery apparatus.

As discussed herein, the partially crimping the annular frame at 520 may comprise radially compressing the implantable prosthetic valve to bring the annular frame to a diameter that is at least substantially equal to a first stop diameter, such as may be defined by a diameter of the crimper aperture.

In some examples, the partially crimping the annular frame at 520 comprises directly engaging the implantable prosthetic valve with the jaws of the crimper assembly.

As disclosed herein, the crimper assembly may comprise a first crimp stop and a second crimp stop that individually or collectively define ranges of motion of an actuator handle of the crimper assembly. In particular, the partially crimping the annular frame at 520 may comprise actuating the actuator handle while the second crimp stop is fixedly coupled relative to the housing of the crimper assembly and while the first crimp stop is operatively coupled to the second crimp stop. Accordingly, in some examples, the method 500 additionally comprises, prior to the partially crimping the annular frame at 520, operatively coupling the first crimp stop to the second crimp stop, such as by inserting the second crimp stop into a second crimp stop receiver of the first crimp stop.

With the first crimp stop operatively coupled to the second crimp stop, the partially crimping the annular frame at 520 can comprise positioning the implantable prosthetic valve within the crimper aperture of the crimper assembly (e.g., as shown in FIG. 13) and actuating the actuator handle to engage the first crimp stop (e.g., as shown in FIG. 14).

In particular, in some examples, the implantable prosthetic valve is positioned within the crimper aperture such that a full valve length of the implantable prosthetic valve is contained within a crimper aperture length of the crimper aperture. As a result, the partially crimping the annular frame at 520 can comprise radially compressing the annular frame evenly across a full length of the annular frame.

At 530, the method 500 can comprise operatively coupling a crimp alignment tool to a distal portion of the valve delivery apparatus to position the crimp alignment tool in an alignment configuration relative to the valve delivery apparatus. In particular, and as described in more detail herein, when the crimp alignment tool is in the alignment configuration, the crimp alignment tool may be utilized to determine and/or establish the target crimping location of the implantable prosthetic valve upon the inflatable balloon.

In some examples, the operatively coupling the crimp alignment tool at 530 comprises engaging the distal portion of the valve delivery apparatus with the crimp alignment tool to position the crimp alignment tool in the alignment configuration. This may be accomplished in any suitable manner, such as by receiving the distal portion of the valve delivery apparatus within the crimp alignment tool such that a stylet of the valve delivery apparatus extends partially within a distal aperture of the crimp alignment tool.

In various examples, the operatively coupling the crimp alignment tool to the valve delivery apparatus at 530 may be performed prior to or subsequent to the partially crimping the annular frame at 520. FIG. 15 may be described as illustrating an example of a configuration of the implantable prosthetic valve and the valve delivery apparatus subsequent to the partially crimping the annular frame at 520 and subsequent to the operatively coupling the crimp alignment tool at 530.

In some examples, the operatively coupling the crimp alignment tool at 530 additionally or alternatively may comprise axially advancing the crimp alignment tool toward the inflatable balloon until the crimp alignment tool is restricted from further axial advancement (such as due to the stylet engaging the crimp alignment tool distal end).

In some examples, the operatively coupling the crimp alignment tool at 530 comprises visually verifying that the crimp alignment tool has reached the alignment configuration. For example, and as discussed above, the crimp alignment tool may comprise a distal alignment indicator configured to provide a visual indication that the crimp alignment tool is in the alignment configuration. Accordingly, in some examples, the method 500 may comprise visually verifying an axial position of the valve delivery apparatus relative to the distal alignment indicator.

As a more specific example, in some examples, visually verifying the axial position of the valve delivery apparatus comprises visually verifying that a component of the valve delivery apparatus (such as a proximal edge of the stylet) is axially aligned with a proximal edge or a distal edge of a window defined in the crimp alignment tool, as shown, for example, in FIG. 15.

Additionally or alternatively, verifying the axial position of the valve delivery apparatus can comprise verifying that a component of the valve delivery apparatus is axially aligned with one or more visual indicia of the crimp alignment tool, as discussed above.

At 540, the method 500 can comprise positioning the implantable prosthetic valve to engage the crimp alignment tool. For example, the positioning the implantable valve to engage the crimp alignment tool at 540 can comprise axially translating the implantable prosthetic valve relative to the valve delivery apparatus such that a distal frame end of the annular frame engages a valve stop of the crimp alignment tool.

In some examples, the positioning the implantable prosthetic valve to engage the crimp alignment tool at 540 is performed subsequent to the partially crimping the annular frame at 520. In particular, when the implantable prosthetic valve is positioned to engage the crimp alignment tool while in the intermediate configuration, the implantable prosthetic valve may remain at a substantially fixed axial position relative to the inflatable balloon while the implantable prosthetic valve is fully crimped onto the inflatable balloon.

Stated differently, in such examples, the positioning the implantable prosthetic valve to engage the crimp alignment tool at 540 is performed with the implantable prosthetic valve in the intermediate configuration, in which the implantable prosthetic valve is substantially fixed in position relative to the inflatable balloon until being deliberately repositioned. In this manner, partially crimping the annular frame to transition the implantable prosthetic valve to the intermediate configuration may facilitate positioning the implantable prosthetic valve to engage the crimp alignment tool such that the implantable prosthetic valve remains in position relative to the crimp alignment tool during a subsequent crimping step, as discussed below.

FIG. 15 further may be described as illustrating an example of a configuration of the implantable prosthetic valve and the valve delivery apparatus subsequent to the partially crimping the annular frame at 520, subsequent to the operatively coupling the crimp alignment tool at 530, and subsequent to the positioning the implantable prosthetic valve at 540.

At 550, the method 500 can comprise fully crimping the annular frame of the implantable prosthetic valve. As discussed herein, the crimper assembly may be utilized to fully crimp the annular frame of the implantable prosthetic valve to bring the implantable prosthetic valve to a delivery configuration. When the implantable prosthetic valve is in the delivery configuration, the annular frame of the implantable prosthetic valve is contracted to a diameter at least substantially equal to a second stop diameter, which is less than the first stop diameter. In some examples, the fully crimping the annular frame at 550 comprises directly engaging the implantable prosthetic valve with the jaws of the crimper assembly.

As discussed herein, the fully crimping the annular frame at 550 may comprise actuating the actuator handle while the second crimp stop is fixedly coupled relative to the housing of the crimper assembly but while the first crimp stop is removed the second crimp stop. Accordingly, in some examples, the method 500 additionally comprises, prior to the fully crimping the annular frame at 550, removing the first crimp stop from the second crimp stop.

In some examples, removing the first crimp stop from the second crimp stop may comprise gripping a grip feature of the initial crimp top to pivot the first crimp stop relative to the second crimp stop and/or to pull the first crimp stop away from the second crimp stop.

With the first crimp stop removed from the second crimp stop, and as shown in FIG. 16, the fully crimping the annular frame at 550 can comprise positioning the implantable prosthetic valve within the crimper aperture of the crimper assembly and actuating the actuator handle to engage the second crimp stop.

In particular, in some examples, the implantable prosthetic valve is positioned within the crimper aperture such that a full valve length of the implantable prosthetic valve is contained within a crimper aperture length of the crimper aperture. As a result, the fully crimping the annular frame at 550 can comprise radially compressing the annular frame evenly across a full length of the annular frame.

In some examples, actuating the actuator handle to engage the second crimp stop once may be insufficient to maintain the implantable prosthetic valve in the delivery configuration after the jaws of the crimper assembly are released from the implantable prosthetic valve. For example, the annular frame of the of the implantable prosthetic valve may be sufficiently stiff and/or resilient that, after compressing the annular frame to the second stop diameter via the fulling crimping the annular frame at 550, the annular frame expands slightly to a diameter that is greater than the second stop diameter after the jaws are released.

Accordingly, in some examples, the method 500 further comprises, subsequent to the actuating the actuator handle to engage the second crimp stop, actuating the actuator handle to release the jaws of the crimper assembly from the implantable prosthetic valve and repeating the actuating the actuator handle to engage the second crimp stop. Stated differently, in such examples, the fully crimping the annular frame at 550 comprises repeatedly crimping the implantable prosthetic valve with the crimper assembly, such as to ensure that the annular frame remains at a diameter substantially equal to the second stop diameter after the jaws are released from the annular frame. In such examples, the fully crimping the annular frame at 550 can comprise bringing the actuator handle in to contact with the second crimp stop any suitable number of times, examples of which include at least two times, at least three times, at least four times, and/or at most five times.

With the implantable prosthetic valve fully crimped upon the inflatable balloon and in the delivery configuration, the valve delivery apparatus may be prepared for insertion into the patient's vasculature, with the possible exception that various components associated with the method 500 may remain coupled to the valve delivery apparatus.

Accordingly, in some examples, the method 500 additionally comprises removing the crimp alignment tool from the valve delivery apparatus and/or removing the stylet of the valve delivery apparatus from the nose piece. For example, FIG. 17 may be described as illustrating a configuration of the implantable prosthetic valve and the valve delivery apparatus subsequent to the fully crimping the annular frame at 550 and with the stylet still coupled to the nose piece, whereas FIG. 18 may be described as illustrating these components subsequent to removing the stylet from the nose piece. Additionally, FIG. 18 illustrates a configuration in which the guide catheter is advanced in a distal direction relative to the configuration of FIG. 17 such that the guide catheter extends proximate to the fully crimped implantable prosthetic valve.

In particular, and with reference to the example of FIGS. 17-18, fully crimping the implantable prosthetic valve 300 to the inflatable balloon 230 may result in the proximal portion 232 of the inflatable balloon 230 being uncovered (as shown in FIG. 17). To prepare the valve delivery apparatus 200 for advancement through the patient's vasculature, the proximal portion 232 of the inflatable balloon 230 may be covered by advancing the guide catheter shaft 214 distally to abut and/or engage the implantable prosthetic valve 300 (as shown in FIG. 18).

As discussed above, various components and/or features of the prosthetic valve preparation assembly 10 may be configured to facilitate advancing the guide catheter shaft 214 over the proximal portion 232 of the inflatable balloon 230. For example, and as discussed above, the engagement between the proximal balloon engagement surfaces 462a/462b and the inflatable balloon 230 may operate to slightly compress and/or to mechanically shape the proximal portion 232 of the inflatable balloon 230 to a profile that is suitably constricted to allow the proximal portion 232 to extend within the inner diameter of the guide catheter shaft 214.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve preparation assembly comprising: an implantable prosthetic valve comprising an annular frame defining a frame central passage and a valve assembly positioned within the frame central passage; a valve delivery apparatus configured to deliver the implantable prosthetic valve through a patient's vasculature to a target implantation site; a crimper assembly for crimping the implantable prosthetic valve around the valve delivery apparatus, wherein the crimper assembly comprises: an actuator handle configured to be manually actuated to crimp the implantable prosthetic valve around the valve delivery apparatus; a second crimp stop that defines a full range of motion of the actuator handle; and a first crimp stop that defines a partial range of motion of the actuator handle and that is configured to be selectively and operatively coupled to the second crimp stop; and a crimp alignment tool configured to facilitate crimping the implantable prosthetic valve relative to an inflatable balloon of the valve delivery apparatus; wherein the crimp alignment tool is configured to engage the implantable prosthetic valve to position the implantable prosthetic valve at a target crimping location relative to the valve delivery apparatus, and wherein the crimp alignment tool is configured to be longitudinally slid onto a distal portion of the valve delivery apparatus to operatively couple the crimp alignment tool to the valve delivery apparatus.

Example 2. The prosthetic valve preparation system of any example herein, particularly example 1, wherein the valve delivery apparatus comprises: a steerable guide catheter comprising a handle portion and a guide catheter shaft extending from the handle portion; a balloon catheter extending through the guide catheter; an inflatable balloon mounted to the balloon catheter; a nose piece mounted at a distal end of the valve delivery apparatus to facilitate advancement of the valve delivery apparatus through the patient's vasculature to the target implantation site; and a stylet configured to be removably coupled to a distal end of the nose piece; and wherein the valve delivery apparatus is configured such that the implantable prosthetic valve is mounted directly to the inflatable balloon prior to introducing the valve delivery apparatus into the patient's vasculature.

Example 3. The prosthetic valve preparation system of any example herein, particularly example 2, wherein the nose piece comprises a nose piece lumen, and wherein the stylet comprises a stylet shaft that is configured to be received within the nose piece lumen to selectively couple the stylet to the nose piece.

Example 4. The prosthetic valve preparation system of any example herein, particularly any one of examples 2-3, further comprising a balloon cover assembly configured to be operatively coupled to the inflatable balloon, the balloon cover assembly comprising: a pair of balloon cover clamshell portions configured to be mounted to opposite sides of the inflatable balloon when the balloon cover assembly is operatively coupled to the inflatable balloon; and a balloon cover sleeve configured to surround the pair of balloon cover clamshell portions to retain the balloon cover clamshell portions relative to the inflatable balloon when the balloon cover assembly is operatively coupled to the inflatable balloon; wherein, when the balloon cover assembly is operatively coupled to the inflatable balloon, the balloon cover sleeve restricts the balloon cover clamshell portions from being removed from the inflatable balloon, and wherein the balloon cover sleeve is configured to slide axially off of the pair of balloon cover clamshell portions to permit the pair of balloon cover clamshell portions to be removed from the inflatable balloon.

Example 5. The prosthetic valve preparation system of any example herein, particularly example 4, wherein the balloon cover assembly is configured to engage the crimp alignment tool such that, when the balloon cover assembly is operatively coupled to the inflatable balloon with the crimp alignment tool operatively coupled to the distal portion of the valve delivery apparatus, the balloon cover assembly restricts the crimp alignment tool from being removed from the distal portion of the valve delivery apparatus.

Example 6. The prosthetic valve preparation system of any example herein, particularly any one of examples 4-5, wherein each balloon cover clamshell portion comprises a proximal balloon engagement surface that is configured to engage the inflatable balloon when the balloon cover assembly is operatively coupled to the inflatable balloon.

Example 7. The prosthetic valve preparation system of any example herein, particularly any one of examples 4-6, wherein the proximal balloon engagement surface is configured to engage the inflatable balloon to mechanically shape at least a portion of the inflatable balloon.

Example 8. The prosthetic valve preparation system of any example herein, particularly any one of examples 1-7, wherein the crimper assembly further comprises: a base; a housing fixedly mounted to the base and defining a crimper assembly central axis; and a plurality of circumferentially arrayed nesting jaws supported within the housing, wherein the jaws are radially moveable within the housing toward and away from the central axis, and wherein the plurality of jaws collectively define a crimper aperture that varies in diameter as the jaws move toward and away from the central axis; wherein the actuator handle is moveable relative to the housing to move the jaws toward and away from the central axis, and wherein the second crimp stop is fixedly coupled relative to the housing during use of the crimper assembly.

Example 9. The prosthetic valve preparation system of any example herein, particularly example 8, wherein the crimper aperture extends along a crimper aperture length, as measured along a direction parallel to the crimper assembly central axis, that is equal to or greater than a valve length of the implantable prosthetic valve.

Example 10. The prosthetic valve preparation system of any example herein, particularly any one of examples 1-9, wherein the first crimp stop comprises a receiver channel that receives an elongate portion of the second crimp stop when the first crimp stop is mechanically coupled to the second crimp stop.

Example 11. The prosthetic valve preparation system of any example herein, particularly any one of examples 1-10, wherein the first crimp stop comprises a grip feature configured to facilitate removal of the first crimp stop from the second crimp stop; and wherein the grip feature comprises one or more of a tab, a lever, a protrusion, and an indentation.

Example 12. The prosthetic valve preparation system of any example herein, particularly any one of examples 1-11, wherein the crimp alignment tool comprises: a catheter receiver configured to receive a distal portion of the valve delivery apparatus to position the crimp alignment tool in an alignment configuration relative to the valve delivery apparatus; a crimp alignment tool distal end configured to engage the distal portion of the valve delivery apparatus to retain the crimp alignment tool in the alignment configuration; and a valve stop positioned in a proximal region of the crimp alignment tool and configured to engage a distal frame end of the annular frame of the implantable prosthetic valve; and wherein the crimp alignment tool is configured such that, when the crimp alignment tool is in the alignment configuration with the crimp alignment tool distal end engaging the distal portion of the valve delivery apparatus, positioning the implantable prosthetic valve such that the distal frame end engages the valve stop operates to position the implantable prosthetic valve at the target crimping location.

Example 13. The prosthetic valve preparation system of any example herein, particularly example 12, wherein the valve delivery apparatus comprises a nose piece and a stylet configured to be removably coupled to a distal end of the nose piece, and wherein the crimp alignment tool is configured such that the stylet engages the crimp alignment tool distal end when the crimp alignment tool is in the alignment configuration.

Example 14. The prosthetic valve preparation system of any example herein, particularly example 13, wherein the nose piece comprises a nose piece lumen, wherein the stylet comprises a stylet shaft that is configured to be received within the nose piece lumen to selectively couple the stylet to the nose piece, wherein the valve delivery apparatus comprises a steerable guide catheter and a balloon catheter extending through the guide catheter, and wherein the stylet shaft is configured to extend within the nose piece lumen and to engage a distal end of the balloon catheter such that, when the crimp alignment tool is in the alignment configuration with the crimp alignment tool distal end engaging the distal portion of the valve delivery apparatus, an axial position of the valve stop coincides with at least a portion of the target crimp location.

Example 15. A crimp alignment tool comprising: a catheter receiver configured to receive a distal portion of a valve delivery apparatus to position the crimp alignment tool in an alignment configuration relative to the valve delivery apparatus; a crimp alignment tool distal end configured to engage the distal portion of the valve delivery apparatus to retain the crimp alignment tool in the alignment configuration; and a valve stop positioned in a proximal region of the crimp alignment tool and configured to engage a distal frame end of an annular frame of an implantable prosthetic valve; and wherein the crimp alignment tool is configured such that, when the crimp alignment tool is in the alignment configuration with the crimp alignment tool distal end engaging the distal portion of the valve delivery apparatus, positioning the implantable prosthetic valve such that the distal frame end engages the valve stop operates to position the implantable prosthetic valve at a target crimping location.

Example 16. The crimp alignment tool of any example herein, particularly example 15, wherein the crimp alignment tool is configured to be longitudinally slid onto the distal portion of the valve delivery apparatus to operatively couple the crimp alignment tool to the valve delivery apparatus.

Example 17. The crimp alignment tool of any example herein, particularly any one of examples 15-16, wherein the crimp alignment tool is configured such that, when the crimp alignment tool is in the alignment configuration, the crimp alignment tool is at least partially restricted from being removed from the valve delivery apparatus.

Example 18. The crimp alignment tool of any example herein, particularly any one of examples 15-17, further comprising a distal alignment indicator configured to provide a visual indication that the crimp alignment tool is in the alignment configuration relative to the valve delivery apparatus, wherein the distal alignment indicator comprises a window that permits visual inspection of the valve delivery apparatus through the crimp alignment tool, and wherein the crimp alignment tool is configured such that, when the crimp alignment tool is in the alignment configuration relative to the valve delivery apparatus, a component of the valve delivery apparatus is axially aligned with one or both of a distal edge of the window and a proximal edge of the window.

Example 19. A method of mounting an implantable prosthetic valve to a valve delivery apparatus, the method comprising: inserting a balloon catheter of the valve delivery apparatus within a frame central passage of the implantable prosthetic valve; partially crimping an annular frame of the implantable prosthetic valve with a crimper assembly; and fully crimping the annular frame of the implantable prosthetic valve.

Example 20. The method of any example herein, particularly example 19, further comprising, subsequent to the partially crimping the annular frame of the implantable prosthetic valve and prior to the fully crimping the annular frame of the implantable prosthetic valve: operatively coupling a crimp alignment tool to a distal portion of the valve delivery apparatus to position the crimp alignment tool in an alignment configuration relative to the valve delivery apparatus; and positioning the implantable prosthetic valve to engage the crimp alignment tool.

Example 21. The method of any example herein, particularly example 20, wherein the valve delivery apparatus comprises a nose piece mounted at a distal end of the valve delivery apparatus and a stylet configured to be removably coupled to a distal end of the nose piece, wherein the crimp alignment tool comprises a window that permits visual inspection of the valve delivery apparatus through the crimp alignment tool, and wherein the method further comprises visually verifying that the stylet is axially aligned with one of a distal edge of the window or a proximal edge of the window.

Example 22. The method of any example herein, particularly any one of examples 20-21, wherein the valve delivery apparatus comprises a nose piece mounted at a distal end of the valve delivery apparatus and a stylet configured to be removably coupled to a distal end of the nose piece, wherein the crimp alignment tool comprises a crimp alignment tool distal end that comprises a distal aperture, wherein the operatively coupling the crimp alignment tool to the distal portion of the valve delivery apparatus comprises engaging the stylet with the crimp alignment tool distal end, and wherein the engaging the stylet comprises inserting the stylet at least partially through the distal aperture.

Example 23. The method of any example herein, particularly any one of examples 20-22, wherein the positioning the implantable prosthetic valve to engage the crimp alignment tool comprises axially translating the implantable prosthetic valve relative to the valve delivery apparatus such that a distal frame end of the annular frame engages a valve stop of the crimp alignment tool.

Example 24. The method of any example herein, particularly any one of examples 19-23, wherein the crimper assembly comprises: an actuator handle configured to be manually actuated to crimp the implantable prosthetic valve around the valve delivery apparatus; a second crimp stop that defines a full range of motion of the actuator handle; and a first crimp stop that defines a partial range of motion of the actuator handle and that is configured to be selectively and operatively coupled to the second crimp stop; wherein the partially crimping the annular frame comprises, with the first crimp stop operatively coupled to the second crimp stop, actuating the actuator handle to engage the first crimp stop, wherein the fully crimping the annular frame comprises actuating the actuator handle to engage the second crimp stop, and wherein the method further comprises, subsequent to the partially crimping the annular frame and prior to the fully crimping the annular frame, removing the first crimp stop from the second crimp stop.

Example 25. A crimper assembly comprising: an actuator handle configured to be manually actuated to crimp an implantable prosthetic valve around a valve delivery apparatus; a second crimp stop that defines a full range of motion of the actuator handle; and a first crimp stop that defines a partial range of motion of the actuator handle and that is configured to be selectively and operatively coupled to the second crimp stop.

Example 26. The crimper assembly of any example herein, particularly example 25, further comprising: a base; a housing fixedly mounted to the base and defining a crimper assembly central axis; and a plurality of circumferentially arrayed nesting jaws supported within the housing, wherein the jaws are radially moveable within the housing toward and away from the central axis, and wherein the plurality of jaws collectively define a crimper aperture that varies in diameter as the jaws move toward and away from the central axis.

Example 27. The crimper assembly of any example herein, particularly example 26, wherein the actuator handle is moveable relative to the housing to move the jaws toward and away from the central axis.

Example 28. The crimper assembly of any example herein, particularly any one of examples 25-27, wherein the second crimp stop is fixedly coupled relative to the housing during use of the crimper assembly.

Example 29. The crimper assembly of any example herein, particularly any one of examples 25-28, wherein, when the first crimp stop is operatively coupled to the second crimp stop, actuating the actuator handle to engage the first crimp stop operates to bring the crimper aperture to a first stop diameter, and wherein, when the first crimp stop is removed from the second crimp stop, actuating the actuator handle to engage the second crimp stop operates to bring the crimper aperture to a second stop diameter that is less than the first stop diameter.

Example 30. The crimper assembly of any example herein, particularly any one of examples 25-29, wherein the crimper aperture extends along a crimper aperture length, as measured along a direction parallel to the crimper assembly central axis, that is equal to or greater than a valve length of the implantable prosthetic valve.

Example 31. The crimper assembly of any example herein, particularly any one of examples 25-30, wherein the first crimp stop comprises a first crimp stop body that defines and terminates at a first crimp stop terminal end, and wherein the crimper assembly is configured such that the actuator handle engages the first crimp stop terminal end during use of the crimper assembly and when the first crimp stop is operatively coupled to the second crimp stop.

Example 32. The crimper assembly of any example herein, particularly any one of examples 25-31, wherein the second crimp stop comprises a second crimp stop body that defines and terminates at a second crimp stop terminal end, and wherein the crimper assembly is configured such that the actuator handle engages the second crimp stop terminal end during use of the crimper assembly and when the first crimp stop is removed from the second crimp stop.

Example 33. The crimper assembly of any example herein, particularly any one of examples 25-32, wherein the first crimp stop comprises a second crimp stop receiver that is configured to receive a portion of the second crimp stop, optionally a second crimp stop terminal end of a second crimp stop body of the second crimp stop, when the first crimp stop is operatively coupled to the second crimp stop.

Example 34. The crimper assembly of any example herein, particularly example 33, wherein the second crimp stop receiver comprises a receiver channel that receives an elongate portion of the second crimp stop when the first crimp stop is operatively coupled to the second crimp stop.

Example 35. The crimper assembly of any example herein, particularly any one of examples 25-34, wherein the first crimp stop comprises a grip feature configured to facilitate removal of the first crimp stop from the second crimp stop.

Example 36. The crimper assembly of any example herein, particularly example 35, wherein the grip feature is configured to be gripped by a user to enable the user to exert a torque on the first crimp stop to remove the first crimp stop from the second crimp stop.

Example 37. The crimper assembly of any example herein, particularly any one of examples 35-36, wherein the grip feature is configured to be gripped by a user to enable the user to pull the first crimp stop away from the second crimp stop.

Example 38. The crimper assembly of any example herein, particularly any one of examples 35-37, wherein the grip feature comprises one or more of a tab, a lever, a protrusion, and an indentation.

Example 39. The crimper assembly of any example herein, particularly any one of examples 25-38, wherein, during use of the crimper assembly, the second crimp stop is coupled to one or both of the base and the housing at a crimp stop attachment location.

Example 40. The crimper assembly of any example herein, particularly example 39, wherein the second crimp stop is not configured to be removed from the crimp stop attachment location without damage to the crimper assembly.

Example 41. The crimper assembly of any example herein, particularly example 39, wherein the second crimp stop is configured to be selectively and repeatedly coupled to and removed from the crimp stop attachment location without damage to the crimper assembly.

Example 42. The crimper assembly of any example herein, particularly any one of examples 39-41, further comprising: a base; a housing fixedly mounted to the base and defining a crimper assembly central axis; and a plurality of circumferentially arrayed nesting jaws supported within the housing, wherein the jaws are radially moveable within the housing toward and away from the central axis, and wherein the plurality of jaws collectively define a crimper aperture that varies in diameter as the jaws move toward and away from the central axis; wherein one or both of the base and the housing comprises a crimp stop receiver that defines the crimp stop attachment location.

Example 43. The crimper assembly of any example herein, particularly example 42, wherein the second crimp stop is configured to be operatively coupled to the crimp stop receiver via one or more of a friction-fit coupling, a snap-fit coupling, a latching coupling, and a keyed coupling.

Example 44. A valve delivery apparatus comprising: a steerable guide catheter comprising a handle portion and a guide catheter shaft extending from the handle portion; a balloon catheter extending through the guide catheter; an inflatable balloon mounted to the balloon catheter; a nose piece mounted at a distal end of the valve delivery apparatus to facilitate advancement of the valve delivery apparatus through a patient's vasculature to a target implantation site; and a stylet configured to be removably coupled to a distal end of the nose piece; wherein the valve delivery apparatus is configured such that an implantable prosthetic valve is mounted directly to the inflatable balloon prior to introducing the valve delivery apparatus into the patient's vasculature.

Example 45. The valve delivery apparatus of any example herein, particularly example 44, wherein the balloon catheter comprises a proximal portion adjacent to the handle portion, a distal portion opposite the proximal portion, and a balloon catheter shaft that extends from the proximal portion and through the handle portion and the guide catheter shaft.

Example 46. The valve delivery apparatus of any example herein, particularly example 45, wherein the inflatable balloon is mounted to the distal portion.

Example 47. The valve delivery apparatus of any example herein, particularly any one of examples 44-46, wherein the guide catheter and the balloon catheter are configured to slide longitudinally relative to one another to facilitate delivery and positioning of the implantable prosthetic valve at the target implantation site.

Example 48. The valve delivery apparatus of any example herein, particularly any one of examples 44-47, wherein the balloon catheter shaft is an outer balloon catheter shaft; and wherein the balloon catheter further comprises an inner balloon catheter shaft that extends from the proximal portion and coaxially through the outer balloon catheter shaft and the inflatable balloon.

Example 49. The valve delivery apparatus of any example herein, particularly example 48, wherein the balloon catheter comprises a fluid passageway that is in fluid communication with an annular space defined between the inner balloon catheter shaft and the outer balloon catheter shaft and that is fluidly connectable to a fluid source for inflating the inflatable balloon.

Example 50. The valve delivery apparatus of any example herein, particularly any one of examples 44-49, wherein the nose piece is configured to move independently of one or both of the guide catheter and the balloon catheter.

Example 51. The valve delivery apparatus of any example herein, particularly any one of examples 44-50, wherein the nose piece comprises a nose piece lumen, and wherein the stylet comprises a stylet shaft that is configured to be received within the nose piece lumen to selectively couple the stylet to the nose piece.

Example 52. The valve delivery apparatus of any example herein, particularly example 51, wherein the stylet shaft is configured to extend within the nose piece lumen and to engage a distal end of the balloon catheter such that, when a crimp alignment tool is in an alignment configuration with a crimp alignment tool distal end engaging a distal portion of the valve delivery apparatus, an axial position of a valve stop of the crimp alignment tool coincides with at least a portion of a target crimp location of the implantable prosthetic valve.

Example The valve delivery apparatus of any example herein, particularly any one of examples 44-52, wherein the stylet is more rigid than the nose piece.

Example 54. The valve delivery apparatus of any example herein, particularly any one of examples 44-53, wherein the implantable prosthetic valve comprises an annular frame defining a frame central passage and a valve assembly positioned within the frame central passage, and wherein the annular frame is radially expandable and contractable to transition the implantable prosthetic valve between a delivery configuration, in which the implantable prosthetic valve is sized for delivery through the patient's vasculature, and an operational configuration, in which the implantable prosthetic valve is sized to be operatively mounted at the target implantation site.

Example 55. The valve delivery apparatus of any example herein, particularly example 54, wherein, when the implantable prosthetic valve is mounted to the inflatable balloon in the delivery configuration, the implantable prosthetic valve is restricted from translating relative to the inflatable balloon.

Example 56. The valve delivery apparatus of any example herein, particularly any one of examples 54-55, wherein the valve delivery apparatus is configured such that inflating the inflatable balloon with the implantable prosthetic valve mounted to the inflatable balloon operates to transition the implantable prosthetic valve from the delivery configuration to the operational configuration.

Example 57. The valve delivery apparatus of any example herein, particularly any one of examples 54-56, wherein the annular frame further is radially expandable and contractable to transition the implantable prosthetic valve to an intermediate configuration defined between the delivery configuration and the operational configuration, and wherein, when the implantable prosthetic valve is mounted to the inflatable balloon in the intermediate configuration, the implantable prosthetic valve engages the inflatable balloon such that the implantable prosthetic valve is at least partially fixed in position relative to the inflatable balloon unless the implantable prosthetic valve is deliberately repositioned by a user.

Example 58. The valve delivery apparatus of any example herein, particularly any one of examples 44-57 in combination with the implantable prosthetic valve.

Example 59. A crimp alignment tool configured to facilitate crimping an implantable prosthetic valve relative to an inflatable balloon of a valve delivery apparatus, wherein the crimp alignment tool is configured to engage the implantable prosthetic valve to position the implantable prosthetic valve at a target crimping location relative to the valve delivery apparatus.

Example 60. The crimp alignment tool of any example herein, particularly example 59, wherein the crimp alignment tool comprises: a catheter receiver configured to receive a distal portion of the valve delivery apparatus to position the crimp alignment tool in an alignment configuration relative to the valve delivery apparatus; a crimp alignment tool distal end configured to engage the distal portion of the valve delivery apparatus to retain the crimp alignment tool in the alignment configuration; and a valve stop positioned in a proximal region of the crimp alignment tool and configured to engage a distal frame end of an annular frame of the implantable prosthetic valve; and wherein the crimp alignment tool is configured such that, when the crimp alignment tool is in the alignment configuration with the crimp alignment tool distal end engaging the distal portion of the valve delivery apparatus, positioning the implantable prosthetic valve such that the distal frame end engages the valve stop operates to position the implantable prosthetic valve at the target crimping location.

Example 61. The crimp alignment tool of any example herein, particularly example 60, wherein the crimp alignment tool is configured to be longitudinally slid onto the distal portion of the valve delivery apparatus to operatively couple the crimp alignment tool to the valve delivery apparatus.

Example 62. The crimp alignment tool of any example herein, particularly any one of examples 59-61, wherein the crimp alignment tool is configured such that, when the crimp alignment tool is in an alignment configuration relative to the valve delivery apparatus, the crimp alignment tool is at least partially restricted from being removed from the valve delivery apparatus.

Example 63. The crimp alignment tool of any example herein, particularly any one of examples 59-62, wherein the crimp alignment tool is configured to engage the distal portion of the valve delivery apparatus in one or more of a friction-fit engagement, a mechanical engagement, a latching engagement, and a snap-fit engagement.

Example 64. The crimp alignment tool of any example herein, particularly any one of examples 59-63, wherein the crimp alignment tool comprises a valve stop positioned in a proximal region of the crimp alignment tool and configured to engage a distal frame end of an annular frame of the implantable prosthetic valve, and wherein the crimp alignment tool terminates at a crimp alignment tool proximal end that defines the valve stop.

Example 65. The crimp alignment tool of any example herein, particularly any one of examples 59-63, wherein the crimp alignment tool comprises a valve stop positioned in a proximal region of the crimp alignment tool and configured to engage a distal frame end of an annular frame of the implantable prosthetic valve, and wherein the crimp alignment tool comprises a stepped interior surface that defines the valve stop.

Example 66. The crimp alignment tool of any example herein, particularly example 65, wherein the crimp alignment tool is configured such that the implantable prosthetic valve is partially received within the crimp alignment tool when the distal frame end engages the valve stop.

Example 67. The crimp alignment tool of any example herein, particularly any one of examples 59-66, wherein the valve delivery apparatus comprises a nose piece and a stylet configured to be removably coupled to a distal end of the nose piece, and wherein the crimp alignment tool is configured such that the stylet engages the crimp alignment tool distal end when the crimp alignment tool is in an alignment configuration relative to the valve delivery apparatus.

Example 68. The crimp alignment tool of any example herein, particularly example 67, wherein the crimp alignment tool is configured such that, when the crimp alignment tool is in an alignment configuration relative to the valve delivery apparatus, an inner diameter of the crimp alignment tool mates with an outer diameter of the stylet.

Example 69. The crimp alignment tool of any example herein, particularly any one of examples 59-68, wherein the crimp alignment tool distal end comprises a distal aperture that receives the distal portion of the valve delivery apparatus when the crimp alignment tool is in an alignment configuration relative to the valve delivery apparatus.

Example 70. The crimp alignment tool of any example herein, particularly example 69, wherein the valve delivery apparatus comprises a nose piece and a stylet configured to be removably coupled to a distal end of the nose piece, and wherein the crimp alignment tool is configured such that the stylet extends at least partially though the distal aperture when the crimp alignment tool is in the alignment configuration.

Example 71. The crimp alignment tool of any example herein, particularly any one of examples 59-70, further comprising a distal alignment indicator configured to provide a visual indication that the crimp alignment tool is in an alignment configuration relative to the valve delivery apparatus.

Example 72. The crimp alignment tool of any example herein, particularly example 71, wherein the distal alignment indicator comprises a window that permits visual inspection of the valve delivery apparatus through the crimp alignment tool.

Example 73. The crimp alignment tool of any example herein, particularly example 72, wherein the crimp alignment tool is configured such that, when the crimp alignment tool is in the alignment configuration relative to the valve delivery apparatus, a component of the valve delivery apparatus is axially aligned with one or both of a distal edge of the window and a proximal edge of the window.

Example 74. The crimp alignment tool of any example herein, particularly any one of examples 71-73, wherein the distal alignment indicator comprises one or more visual indicia such that, when the crimp alignment tool is in the alignment configuration relative to the valve delivery apparatus, a component of the valve delivery apparatus is axially aligned with the visual indicia.

Example 75. The crimp alignment tool of any example herein, particularly example 74, wherein the visual indicia comprise one or more of a mark, a colored mark, a printed mark, an embossed mark, a debossed mark, a line, and a symbol.

Example 76. A balloon cover assembly comprising: a pair of balloon cover clamshell portions configured to be mounted to opposite sides of an inflatable balloon when the balloon cover assembly is operatively coupled to the inflatable balloon; and a balloon cover sleeve configured to surround the pair of balloon cover clamshell portions to retain the balloon cover clamshell portions relative to the inflatable balloon when the balloon cover assembly is operatively coupled to the inflatable balloon; wherein, when the balloon cover assembly is operatively coupled to the inflatable balloon, the balloon cover sleeve restricts the balloon cover clamshell portions from being removed from the inflatable balloon, and wherein the balloon cover sleeve is configured to slide axially off of the pair of balloon cover clamshell portions to permit the pair of balloon cover clamshell portions to be removed from the inflatable balloon.

Example 77. The balloon cover assembly of any example herein, particularly example 76, wherein each balloon cover clamshell portion comprises a proximal balloon engagement surface that is configured to engage the inflatable balloon when the balloon cover assembly is operatively coupled to the inflatable balloon.

Example 78. The balloon cover assembly of any example herein, particularly example 77, wherein the proximal balloon engagement surface is configured to mechanically shape at least a portion of the inflatable balloon.

Example 79. The balloon cover assembly of any example herein, particularly any one of examples 77-78, wherein the proximal balloon engagement surface is configured to engage the inflatable balloon to restrict the balloon cover clamshell portion from translating axially relative to the inflatable balloon when the balloon cover assembly is operatively coupled to the inflatable balloon.

Example 80. The balloon cover assembly of any example herein, particularly any one of examples 77-79, wherein the proximal balloon engagement surface comprises one or more locking ribs that extend radially toward the inflatable balloon when the balloon cover assembly is operatively coupled to the inflatable balloon.

Example 81. The balloon cover assembly of any example herein, particularly example 80, wherein the locking ribs of each proximal balloon engagement surface of the pair of balloon cover clamshell portions are staggered relative to one another such that the locking ribs of the proximal balloon engagement surfaces of each of the pair of balloon cover clamshell portions are interleaved with one another when the balloon cover assembly is operatively coupled to the inflatable balloon.

Example 82. The balloon cover assembly of any example herein, particularly any one of examples 77-81, wherein the respective proximal balloon engagement surfaces of each of the pair of balloon cover clamshell portions are configured to engage one another to mechanically retain the balloon cover clamshell portions against one another when the balloon cover assembly is operatively coupled to the inflatable balloon.

Example 83. The balloon cover assembly of any example herein, particularly any one of examples 76-80, wherein the pair of balloon cover clamshell portions are configured to engage one another when the balloon cover assembly is operatively coupled to the inflatable balloon.

Example 84. The balloon cover assembly of any example herein, particularly any one of examples 76-83, wherein the pair of balloon cover clamshell portions are configured to abut one another when the balloon cover assembly is operatively coupled to the inflatable balloon.

Example 85. The balloon cover assembly of any example herein, particularly any one of examples 76-84, wherein the pair of balloon cover clamshell portions are configured to mate with one another when the balloon cover assembly is operatively coupled to the inflatable balloon.

Example 86. The balloon cover assembly of any example herein, particularly any one of examples 76-85, wherein the balloon cover assembly is configured to engage a crimp alignment tool that is configured to receive a distal portion of a valve delivery apparatus such that, when the balloon cover assembly is operatively coupled to the inflatable balloon with the crimp alignment tool operatively coupled to the distal portion of the valve delivery apparatus, the balloon cover assembly restricts the crimp alignment tool from being removed from the distal portion of the valve delivery apparatus.

Example 87. The balloon cover assembly of any example herein, particularly example 86, wherein the crimp alignment tool comprises a clamshell engagement feature, and wherein each balloon cover clamshell portion comprises a crimp alignment tool engagement feature that engages the clamshell engagement feature when the balloon cover assembly operatively engages the crimp alignment tool.

Example 88. The balloon cover assembly of any example herein, particularly example 87, wherein the clamshell engagement feature comprises an annular groove; and wherein each crimp alignment tool engagement feature comprises an engagement tooth that is configured to be received within a portion of the annular groove to restrict the crimp alignment tool from translating axially relative to the balloon cover assembly when the balloon cover assembly operatively engages the crimp alignment tool.

Example 89. A method of mounting an implantable prosthetic valve to a valve delivery apparatus, the method comprising: inserting a balloon catheter of the valve delivery apparatus within a frame central passage of the implantable prosthetic valve; partially crimping an annular frame of the implantable prosthetic valve with a crimper assembly; operatively coupling a crimp alignment tool to a distal portion of the valve delivery apparatus to position the crimp alignment tool in an alignment configuration relative to the valve delivery apparatus; positioning the implantable prosthetic valve to engage the crimp alignment tool; and fully crimping the annular frame of the implantable prosthetic valve; and wherein one or more of: the crimper assembly is the crimper assembly of any example herein, particularly any one of examples 25-43; the valve delivery apparatus is the valve delivery apparatus of any example herein, particularly any one of examples 43-57; and the crimp alignment tool is the crimp alignment tool of any example herein, particularly any one of examples 58-74.

Example 90. The method of any example herein, particularly example 89, wherein the partially crimping the annular frame of the implantable prosthetic valve comprises radially compressing the implantable prosthetic valve to bring the annular frame to a diameter at least substantially equal to a first stop diameter, and wherein the fully crimping the annular frame of the implantable prosthetic valve comprises radially compressing the implantable prosthetic valve to bring the annular frame to a diameter at least substantially equal to a second stop diameter that is less than the first stop diameter.

Example 91. The method of any example herein, particularly example 90, wherein the partially crimping the annular frame comprises transitioning the implantable prosthetic valve to an intermediate configuration in which the implantable prosthetic valve engages an inflatable balloon of the valve delivery apparatus such that the implantable prosthetic valve is at least partially fixed in position relative to the inflatable balloon unless the implantable prosthetic valve is deliberately repositioned by a user, and wherein the positioning the implantable prosthetic valve to engage the crimp alignment tool is performed with the implantable prosthetic valve in the intermediate configuration.

Example 92. The method of any example herein, particularly any one of examples 89-91, wherein the valve delivery apparatus further comprises: a balloon cover assembly operatively coupled to an inflatable balloon of the valve delivery apparatus, the balloon cover assembly comprising: a pair of balloon cover clamshell portions mounted to opposite sides of the inflatable balloon; and a balloon cover sleeve surrounding the pair of balloon cover clamshell portions to retain the balloon cover clamshell portions relative to the inflatable balloon; and wherein the method further comprises, prior to the inserting the balloon catheter within the frame central passage, removing the balloon cover assembly from the inflatable balloon.

Example 93. The method of any example herein, particularly example 92, wherein the removing the balloon cover assembly from the inflatable balloon comprises: removing the balloon cover sleeve from the pair of balloon cover clamshell portions; and subsequent to the removing the balloon cover sleeve, removing the pair of balloon cover clamshell portions from the inflatable balloon.

Example 94. The method of any example herein, particularly any one of examples 89-93, wherein the operatively coupling the crimp alignment tool to the distal portion of the valve delivery apparatus comprises engaging the distal portion of the valve delivery apparatus with the crimp alignment tool to position the crimp alignment tool in the alignment configuration.

Example 95. The method of any example herein, particularly any one of examples 89-94, wherein the operatively coupling the crimp alignment tool to the distal portion of the valve delivery apparatus comprises axially advancing the crimp alignment tool toward the inflatable balloon until the crimp alignment tool is restricted from further axial advancement.

Example 96. The method of any example herein, particularly any one of examples 89-95, wherein the operatively coupling the crimp alignment tool to the distal portion of the valve delivery apparatus comprises engaging a stylet of the valve delivery apparatus with a crimp alignment tool distal end of the crimp alignment tool.

Example 97. The method of any example herein, particularly example 96, wherein the crimp alignment tool distal end comprises a distal aperture; and wherein the engaging the stylet comprises inserting the stylet at least partially through the distal aperture.

Example 98. The method of any example herein, particularly any one of examples 89-97, wherein the crimp alignment tool comprises a distal alignment indicator configured to provide a visual indication that the crimp alignment tool is in the alignment configuration relative to the valve delivery apparatus, and wherein the operatively coupling the crimp alignment tool to the distal portion of the valve delivery apparatus comprises visually verifying an axial position of the valve delivery apparatus relative to the distal alignment indicator.

Example 99. The method of any example herein, particularly example 98, wherein the distal alignment indicator comprises a window that permits visual inspection of the valve delivery apparatus through the crimp alignment tool, and wherein the visually verifying the axial position comprises verifying that a component of the valve delivery apparatus is axially aligned with one or both of a distal edge of the window and a proximal edge of the window.

Example 100. The method of any example herein, particularly example 99, wherein the component of the valve delivery apparatus is a proximal edge of a stylet of the valve delivery apparatus.

Example 101. The method of any example herein, particularly any one of examples 98-100, wherein the distal alignment indicator comprises one or more visual indicia, and wherein the verifying the axial position comprises verifying that a component of the valve delivery apparatus is axially aligned with the visual indicia.

Example 102. The method of any example herein, particularly any one of examples 89-101, further comprising, prior to the partially crimping the annular frame of the implantable prosthetic valve, operatively coupling a first crimp stop of the crimper assembly to a second crimp stop of the crimper assembly.

Example 103. The method of any example herein, particularly example 102, wherein the first crimp stop comprises a second crimp stop receiver that is configured to receive a portion of the second crimp stop, and wherein the operatively coupling the first crimp stop to the second crimp stop comprises inserting the second crimp stop into the second crimp stop receiver.

Example 104. The method of any example herein, particularly any one of examples 89-103, wherein the partially crimping the annular frame of the implantable prosthetic valve comprises directly engaging the implantable prosthetic valve with jaws of the crimper assembly.

Example 105. The method of any example herein, particularly any one of examples 89-104, wherein the partially crimping the annular frame of the implantable prosthetic valve comprises, with a second crimp stop of the crimper assembly fixedly coupled relative to a housing of the crimper assembly and with a first crimp stop of the crimper assembly operatively coupled to the second crimp stop: positioning the implantable prosthetic valve within a crimper aperture of the crimper assembly; and actuating an actuator handle of the crimper assembly to engage the first crimp stop.

Example 106. The method of any example herein, particularly example 105, wherein the positioning the implantable prosthetic valve within the crimper aperture comprises positioning such that a full valve length of the implantable prosthetic valve is contained within a crimper aperture length of the crimper aperture.

Example 107. The method of any example herein, particularly any one of examples 89-106, wherein the positioning the implantable prosthetic valve relative to the crimp alignment tool comprises axially translating the implantable prosthetic valve relative to the valve delivery apparatus such that a distal frame end of the annular frame engages a valve stop of the crimp alignment tool.

Example 108. The method of any example herein, particularly any one of examples 89-107, further comprising, prior to the fully crimping the annular frame of the implantable prosthetic valve, removing a first crimp stop of the crimper assembly from a second crimp stop of the crimper assembly.

Example 109. The method of any example herein, particularly example 108, wherein the first crimp stop comprises a grip feature configured to facilitate removal of the first crimp stop from the second crimp stop, and wherein the removing the first crimp stop from the second crimp stop comprises gripping the grip feature to one or both of: pivot the first crimp stop relative to the second crimp stop; and pull the first crimp stop away from the second crimp stop.

Example 110. The method of any example herein, particularly any one of examples 89-109, wherein the fully crimping the annular frame of the implantable prosthetic valve comprises directly engaging the implantable prosthetic valve with jaws of the crimper assembly.

Example 111. The method of any example herein, particularly any one of examples 89-110, wherein the fully crimping the annular frame of the implantable prosthetic valve comprises, with a second crimp stop of the crimper assembly fixedly coupled relative to a housing of the crimper assembly and with a first crimp stop of the crimper assembly removed from the second crimp stop: positioning the implantable prosthetic valve within a crimper aperture of the crimper assembly; and actuating an actuator handle of the crimper assembly to engage the second crimp stop.

Example 112. The method of any example herein, particularly example 111, wherein the positioning the implantable prosthetic valve within the crimper aperture comprises positioning such that a full valve length of the implantable prosthetic valve is contained within a crimper aperture length of the crimper aperture.

Example 113. The method of any example herein, particularly any one of examples 111-112, further comprising, subsequent to the actuating the actuator handle to engage the second crimp stop: actuating the actuator handle to release a plurality of jaws of the crimper assembly from the implantable prosthetic valve; and repeating the actuating the actuator handle to engage the second crimp stop and the actuating the actuator handle to release the jaws from the implantable prosthetic valve.

Example 114. The method of any example herein, particularly example 113, wherein the repeating comprises repeating such that the actuating the actuator handle to engage the second crimp stop is performed one or more of at least two times, at least three times, at least four times, and at most five times.

Example 115. The method of any example herein, particularly any one of examples 89-114, further comprising, subsequent to the fully crimping the annular frame, removing the crimp alignment tool from the valve delivery apparatus.

Example 116. The method of any example herein, particularly any one of examples 89-115, further comprising, subsequent to the removing the crimp alignment tool from the valve delivery apparatus, removing a stylet of the valve delivery apparatus from a nose piece of the valve delivery apparatus.

Example 117. The prosthetic valve preparation assembly of any example herein, particularly any one of examples 1-14, wherein one or more components of the valve preparation assembly are sterilized.

Example 118. A method comprising: sterilizing one or more components of the valve preparation assembly of any example herein, particularly any one of examples 1-14.

Example 119. The crimp alignment tool of any example herein, particularly any one of examples 15-18, wherein the crimp alignment tool is sterilized.

Example 120. A method comprising: sterilizing the crimp alignment tool of any example herein, particularly any one of examples 15-18.

Example 121. The crimper assembly of any example herein, particularly any one of examples 25-43, wherein one or more components of the crimper assembly are sterilized.

Example 122. A method comprising: sterilizing one or more components of the crimper assembly of any example herein, particularly any one of examples 25-43.

Example 123. The valve delivery apparatus of any example herein, particularly any one of examples 44-58, wherein one or more components of the valve delivery apparatus are sterilized.

Example 124. A method comprising: sterilizing one or more components of the valve delivery apparatus of any example herein, particularly any one of examples 44-58.

Example 125. The crimp alignment tool of any example herein, particularly any one of examples 59-75, wherein the crimp alignment tool is sterilized.

Example 126. A method comprising: sterilizing the crimp alignment tool of any example herein, particularly any one of examples 59-75.

Example 127. The balloon cover assembly of any example herein, particularly any one of examples 76-88, wherein one or more components of the balloon cover assembly are sterilized.

Example 128. A method comprising: sterilizing one or more components of the balloon cover assembly of any example herein, particularly any one of examples 76-88.

The features described herein with regard to any example can be combined with other features described in any one or more of the other examples, unless otherwise stated. For example, any one or more of the features of one prosthetic valve preparation system can be combined with any one or more features of another prosthetic valve preparation system. As another example, any one or more features of one crimper assembly can be combined with any one or more features of another crimper assembly.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A crimper assembly comprising:
an actuator handle configured to be manually actuated to crimp an implantable prosthetic valve around a valve delivery apparatus;
a second crimp stop that defines a full range of motion of the actuator handle; and
a first crimp stop that defines a partial range of motion of the actuator handle and that is configured to be selectively and operatively coupled to the second crimp stop.

2. The crimper assembly of claim 1, further comprising:
a base;
a housing fixedly mounted to the base and defining a crimper assembly central axis; and
a plurality of circumferentially arrayed nesting jaws supported within the housing, wherein the jaws are radially moveable within the housing toward and away from the central axis, and wherein the plurality of jaws collectively define a crimper aperture that varies in diameter as the jaws move toward and away from the central axis.

3. The crimper assembly of claim 2, wherein the actuator handle is moveable relative to the housing to move the jaws toward and away from the central axis.

4. The crimper assembly of claim 1, wherein the second crimp stop is fixedly coupled relative to the housing during use of the crimper assembly.

5. The crimper assembly of claim 1, wherein, when the first crimp stop is operatively coupled to the second crimp stop, actuating the actuator handle to engage the first crimp stop operates to bring the crimper aperture to a first stop diameter, and
wherein, when the first crimp stop is removed from the second crimp stop, actuating the actuator handle to engage the second crimp stop operates to bring the crimper aperture to a second stop diameter that is less than the first stop diameter.

6. The crimper assembly of claim 1, wherein the crimper aperture extends along a crimper aperture length, as measured along a direction parallel to the crimper assembly central axis, that is equal to or greater than a valve length of the implantable prosthetic valve.

7. The crimper assembly of claim 1, wherein the first crimp stop comprises a first crimp stop body that defines and terminates at a first crimp stop terminal end, and wherein the crimper assembly is configured such that the actuator handle engages the first crimp stop terminal end during use of the crimper assembly and when the first crimp stop is operatively coupled to the second crimp stop.

8. The crimper assembly of claim 1, wherein the second crimp stop comprises a second crimp stop body that defines and terminates at a second crimp stop terminal end, and wherein the crimper assembly is configured such that the actuator handle engages the second crimp stop terminal end during use of the crimper assembly and when the first crimp stop is removed from the second crimp stop.

9. The crimper assembly of claim 1, wherein the first crimp stop comprises a second crimp stop receiver that is configured to receive a portion of the second crimp stop, optionally a second crimp stop terminal end of a second crimp stop body of the second crimp stop, when the first crimp stop is operatively coupled to the second crimp stop.

10. The crimper assembly of claim 9, wherein the second crimp stop receiver comprises a receiver channel that receives an elongate portion of the second crimp stop when the first crimp stop is operatively coupled to the second crimp stop.

11. The crimper assembly of claim 1, wherein the first crimp stop comprises a grip feature configured to facilitate removal of the first crimp stop from the second crimp stop.

12. The crimper assembly of claim 11, wherein the grip feature is configured to be gripped by a user to enable the user to exert a torque on the first crimp stop to remove the first crimp stop from the second crimp stop.

13. The crimper assembly of claim 11, wherein the grip feature is configured to be gripped by a user to enable the user to pull the first crimp stop away from the second crimp stop.

14. The crimper assembly of claim 11, wherein the grip feature comprises one or more of a tab, a lever, a protrusion, and an indentation.

15. The crimper assembly of claim 1, wherein, during use of the crimper assembly, the second crimp stop is coupled to one or both of the base and the housing at a crimp stop attachment location.

16. The crimper assembly of claim 15, wherein the second crimp stop is not configured to be removed from the crimp stop attachment location without damage to the crimper assembly.

17. The crimper assembly of claim 15, wherein the second crimp stop is configured to be selectively and repeatedly coupled to and removed from the crimp stop attachment location without damage to the crimper assembly.

18. The crimper assembly of claim 15, further comprising:
a base;
a housing fixedly mounted to the base and defining a crimper assembly central axis; and
a plurality of circumferentially arrayed nesting jaws supported within the housing, wherein the jaws are radially moveable within the housing toward and away from the central axis, and wherein the plurality of jaws collectively define a crimper aperture that varies in diameter as the jaws move toward and away from the central axis;
wherein one or both of the base and the housing comprises a crimp stop receiver that defines the crimp stop attachment location.

19. A method of mounting an implantable prosthetic valve to a valve delivery apparatus, the method comprising:
inserting a balloon catheter of the valve delivery apparatus within a frame central passage of the implantable prosthetic valve;
partially crimping an annular frame of the implantable prosthetic valve with a crimper assembly;
operatively coupling a crimp alignment tool to a distal portion of the valve delivery apparatus to position the crimp alignment tool in an alignment configuration relative to the valve delivery apparatus;
positioning the implantable prosthetic valve to engage the crimp alignment tool; and
fully crimping the annular frame of the implantable prosthetic valve.

20. The method of claim 19, wherein the crimper assembly comprises:
an actuator handle configured to be manually actuated to crimp the implantable prosthetic valve around the valve delivery apparatus;
a second crimp stop that defines a full range of motion of the actuator handle; and
a first crimp stop that defines a partial range of motion of the actuator handle and that is configured to be selectively and operatively coupled to the second crimp stop;
wherein the partially crimping the annular frame comprises, with the first crimp stop operatively coupled to the second crimp stop, actuating the actuator handle to engage the first crimp stop,
wherein the fully crimping the annular frame comprises actuating the actuator handle to engage the second crimp stop, and
wherein the method further comprises, subsequent to the partially crimping the annular frame and prior to the fully crimping the annular frame, removing the first crimp stop from the second crimp stop.
